(19) United States
(12) Patent Application Publication
(10) Pub. No.: US 2003/0157705 A1
(43) Pub. Date: Aug. 21, 2003

(54) Engineered recombinant molecule that regulates humoral and cellular effector functions of the immune system

(76) Inventors: William L. Fodor, Madison, CT (US); Maryellen Pizzolato, Old Saybrook, CT (US)

Correspondence Address: Mark Farber, c/o Alexion Pharmaceuticals, Inc., 352 Knotter Drive, Cheshire, CT, 06410, US

(21) Appl. No.: 09/928,267

(22) Filed: Aug. 10, 2001

(51) Int. Cl. A01K 67/027; C12N 9/99; C12N 5/06

(52) U.S. Cl. 435/325; 800/17; 435/184

(57) ABSTRACT

Recombinant chimeric molecules that include at least a domain capable of regulating the humoral effector functions of the immune system and another domain capable of regulating the cellular effector functions of the immune system are provided. Recombinant DNA constructs having DNA sequences encoding the above mentioned chimeric proteins are provided. Cloning vectors incorporating the above DNA constructs and cells transformed with the vectors and host cells containing such vectors are also provided. Transgenic cells, tissues, organs, and animals incorporating the above-mentioned chimeric molecules are provided.

### TECHNICAL FIELD

Chimeric proteins capable of conferring resistance to humoral and cellular mechanisms of immune attack and more particularly chimeric proteins having at least a domain derived from a complement inhibitor protein and a domain derived from a T-Cell inhibitor protein are provided. DNA constructs encoding such chimeric proteins and methods of preparing such chimeric proteins are disclosed. Methods of using such chimeric proteins, including in the prevention or treatment of rejection of xenotransplants are described.

### BACKGROUND

Chimeric proteins, also referred to in the art as fusion proteins, are hybrid proteins which combine at least parts of two or more precursor proteins or peptides. Chimeric proteins may be produced by recombinant technology, i.e. by fusing at least a part of the coding sequence of one gene to at least a part of the coding sequence of another gene. The fused gene may then be used to transform a suitable organism which then expresses the fusion protein.

It is known in the art that T cells, also called T lymphocytes, are a part of the vertebrate immune system. T cells recognize foreign pathogens (such as bacteria, viruses, or parasites), tissues, and/or organs, and help the immune system process them (causing what is referred to in the art as a cellular immune response), generally clearing the pathogens from the body. T cell activation is not only dependent on antigen recognition, but also on engagement of costimulatory molecules found on antigen presenting cells (APCs). The costimulatory signal that determines whether antigen recognition leads to full T cell activation or to T cell unresponsiveness, i.e. anergy, is that generated by the interaction of CD28 on the T cells with B7 on the APCs; see for example Harding et al., Nature (1992) 356;607 who demonstrated in vitro that crosslinking of the CD28 molecule can rescue T cells from becoming anergic. It is further known that both B7-1 (CD80) and B7-2 (CD86) molecules on APCs provide critical costimulatory signals in T cell activation through their binding with the CD28 molecule on the T cell, and, moreover, that antigens presented in the absence of such costimulatory signals results in T cell anergy.

It is also know in the art that the complement system, (known in the art to be part of the humoral immune system) is an interaction of at least 25 plasma proteins and membrane cofactors which act in a multistep, multiprotein cascade sequence in conjunction with other immunological systems of the body to defend against intrusion of foreign cells and viruses. Complement components achieve their immune defensive functions by interacting in a series of intricate but precise enzymatic cleavage and membrane binding events. The resulting complement cascade leads to the production of products with opsonic, immunoregulatory, and lytic functions.

CD59 is known to be the archetypical inhibitor of part of the complement system known as the C5b-9 membrane attack complex (MAC). When activated and not inhibited the C5b-9 MAC can cause potentially deleterious cell activation including cell lysis. CD59 is a human glycoprotein, the nucleotide and amino acid sequences for which are set forth in FIG. 2E1. CD59 is found associated with the membranes of cells including human erythrocytes, lymphocytes, and vascular endothelial cells. It serves to prevent assembly of functional MAC's and thus protects cells from complement-mediated activation and/or lysis and is tethered to the outside of the cell membrane by a glycosyl-phosphatidylinositol (GPI) anchor. See, for example, Sims et al., U.S. Pat. No. 5,135,916.

Both humoral and cellular defense mechanisms mediate the rejection of transplanted cells, tissues, and organs during xenotransplantation. The survival of organs and tissues during xenotransplantation requires multiple immunosuppressive strategies to inhibit antibody reactivity, complement activation, and cellular rejection.

Bi-functional complement inhibitors including fusion proteins constructed from the C3 family of inhibitor proteins (such DAF or CD55) and the C5b-9 family of inhibitor proteins (such as CD59) are known. See U.S. Pat. Nos. 5,847,082, 5,624,837, and 5,627,264. It has been demonstrated that the CD59 moiety in a DAF-CD59 chimeric molecule functions to inhibit MAC when expressed membrane proximal and anchored through its endogenous GPI linkage. See Fodor et al., (J. Immunol., 155:4135, 1995).

Various techniques have been investigated to regulate T-cell interactions and immune responses mediated by such interactions. It is known that CTLA4 is a T-cell surface receptor that associates with the B7-1 (CD80) and B7-2 (CD86) molecules which are expressed on antigen-presenting cells. See for example Hancock et al. “Comparative Analysis of B7-1 and B7-2 Co-Stimulatory Ligands: Expression and Function” J. Exp. Med., 180:631, 1994. It is further known that this association establishes the molecular basis for an important T Cell co-stimulatory pathway, the primary function of which is to induce T-cell cytokine production and proliferation following exposure to antigen. See for example Linsley et al., J. Exp. Med. 173:721-730, 1991. U.S. Pat. No. 5,434,131 identifies the CTLA4 receptor as a ligand for the B7 antigen and discloses methods for using soluble fusion proteins to regulate immune responses, including T-cell interactions. U.S. Pat. No. 5,773,253 provides CTLA4 mutant molecules as ligands for the B7 antigen and methods for expressing the mutant molecules as soluble functional molecules which regulate T-cell interactions. U.S. Pat. Nos. 5,844,095 and 5,851,795 describe methods of expressing CTLA4 as an immunoglobulin fusion protein, methods of preparing hybrid CTLA4 fusion proteins and methods of using the soluble fusion proteins, fragments and derivatives thereof, to regulate cellular immune responses and T-cell interactions. U.S. Pat. No. 5,869,050 discloses methods of blocking T-cell activation using anti-B7 monoclonal antibodies to overcome allograft transplant rejection and/or graft versus host disease, as well as to prevent or treat rheumatoid arthritis.

However, no single molecule exists today which can be used in the prevention or treatment of both humoral and cellular rejection of xenotransplants. No such molecules exist that when expressed provide the cell with both protection from human serum complement and inhibit T-cell activation. Such a molecule would be particularly advantageous in the production of transgenic animals. Microinjection of recombinant DNA into the pronuclei of animal ova for generating transgenic animals is known. However, since this technology is dependent on random integration of DNA, it is a complex procedure to achieve targeted cellular expression of two distinct heterologous proteins by the simultaneous microinjection of their respective DNAs (such as would be required if CTLA4 inhibitory activity and CD59 inhibitory activity were to be achieved through the use of individual entities.)

Moreover, as described above, currently the soluble form of CTLA4 or CTLA4IG fusion proteins are used to regulate cellular immune responses and T-cell interactions. Therefore, it would be of additional advantage if the CTLA4 moiety could bind endogenously expressed B7-1 and B7-2 molecules and cis and block the co-stimulation necessary for engagement of human CD28 expressed on T-cells, thereby protecting the xenotransplanted porcine cell from the human cellular immune response by rendering the human T-cell unresponsive to the porcine target cell.

### SUMMARY

Recombinant chimeric molecules that include at least a domain capable of regulating the humoral effector functions of the immune system and another domain capable of regulating the cellular effector functions of the immune system now surprisingly have been engineered. Suitable domains capable of regulating the humoral effector functions of the immune system include complement inhibitory domains, such as a C5b-9 and/or C3 inhibitory domains. Suitable domains capable of regulating the cellular effector functions of the immune system include T Cell inhibitory domains. In one embodiment, a membrane bound chimeric molecule which includes functional domains derived from CTLA4 and CD59 is provided. In another embodiment, a membrane bound chimeric molecule which includes functional domains derived from CTLA and DAF is provided.

Recombinant DNA constructs having DNA sequences encoding the above mentioned chimeric proteins are provided. Cloning vectors incorporating the above DNA constructs and cells transformed with the vectors and host cells containing such vectors are also provided. Transgenic cells, tissues, organs, and animals incorporating the above-mentioned chimeric molecules are provided.

Methods for preparing a DNA construct including a DNA sequence encoding a CD59 inhibitory domain operably linked to a DNA sequence encoding a T Cell inhibitory domain are provided. Also provided are methods of manufacturing the above described chimeric proteins by transforming a cell with a suitable cloning vector including a DNA construct encoding the chimeric protein, and expressing the gene such that the resulting protein on the cell membrane.

### BRIEF DESCRIPTION OF THE DRAWINGS

FIG. 1 depicts a diagramatic representation of the recombinant chimeric molecules.

FIG. 2A(1) depicts the DNA used in the cloning of porcine CTLA4—human CD59 chimeric molecules.

FIG. 2A(2) depicts the amino acid sequence of porcine CTLA4—human CD59 chimeric molecules.

FIG. 2B(1) depicts the DNA used in the cloning of human CTLA4—human CD59 chimeric molecules.

FIG. 2B(2) depicts the amino acid sequence of human CTLA4—human CD59 chimeric molecules.

FIG. 2C(1) depicts the DNA sequence of porcine CTLA4.

FIG. 2C(2) depicts the amino acid sequence of porcine CTLA4.

FIG. 2D(1) depicts the DNA sequence of human CTLA4.

FIG. 2D(2) depicts the amino acid sequence of human CTLA4.

FIG. 2E(1) depicts the DNA sequence of human CD59.

FIG. 2E(2) depicts the amino acid sequence of human CD59.

FIG. 3 are florescence activated cell sorting (FACS) profiles that demonstrate the expression of the CTLA4 and CD59 domains of the chimeric molecules on the cell surface of transduced PAECs. Cell surface expression of hCC. Drug resistant populations of porcine aortic endothelial cells (PAECs) transduced with pBABEhCTLA4-hCD59 or pBABE vector alone were assayed for expression of CD59 and CTLA4. Briefly, cells were incubated with 10 ug/ml anti CTLA4, ANC152.2 (Ancell, Bayport, Minn.) or with 10 ug/ml of either of the anti-CD59 antibodies, BRA10G or MEM43 (Biodesign, Kennebunk, Me.), for 30 min., at 4C, in 0.1 ml of Dulbecco's PBS (DPBS) containing 1% Fetal Bovine Serum (FBS) or Bovine Serum Albumin (BSA). Cells were washed with DPBS before incubation with FITC conjugated antibodies to mouse IgG (Zymed, So. San Francisco, Calif.). Cells were analyzed on a Becton Dickenson FACSORT (Becton Dickenson, Franklin Lakes, N.J.). Phosphatidyl inositol phospholipase C (PI-PLC) enzymatically cleaves gpi-linked molecules from the surface of cells and therefore should cleave the hCTLA4-hCD59 and pCTLA4-hCD59 molecules from the transduced PAECs.

FIG. 4 depicts a FACS analysis that shows the phosphatidyl inositol phospholipase C (PI-PLC) mediated removal of CTLA4 and CD59 domains from transduced PAECs. PI-PLC removal of CC. FACS analysis was performed as described above in FIG. 3 with the following exceptions. Drug resistant populations of porcine aortic endothelial cells (PAECs) transduced with pBABEhCTLA4-hCD59, or pBABEpCTLA4-hCD59 or pBABE vector alone were assayed for expression of CD59. Briefly, cells were incubated with 10ug/ml of either of the anti-CD59 antibodies, BRA10G or MEM43 (Biodesign, Kennebunk, Me.), for 30 min., at 4C, in 0.1 ml of Dulbecco's PBS (DPBS) containing 1% Fetal Bovine Serum (FBS) or Bovine Serum Albumin (BSA). Cells were washed with DPBS before incubation with FITC conjugated antibodies to mouse IgG (Zymed, So. San Francisco, Calif.). In addition, an aliquot of the cell lines were treated with PIPLC, (Boehringer Manniheim GmbH, Indianapolis, Ind.) at 1 U/ml for 1 hour at 37C, prior to antibody incubations and FACS analysis on a Becton Dickenson FACSORT (Becton Dickenson, Franklin Lakes, N.J.).

FIG. 5 depicts the results of cell killing experiments in which porcine aortic endothelia cells (PAECs) that express the chimeric molecules are protected from human serum-induced complement-mediated cell lysis. 5×10 3 vector control or hCTLA4hCD59 cells were seeded into the wells of a flat bottom 96 well plate 24 hours ahead of time. Adherent cells were washed twice using HBSS containing 1% BSA. Cells were sensitized by incubating with a polyclonal anti-PAEC antibody (Cocalico, Reamstown, Pa.), followed by incubation with the intracellular dye, Calcein AM (Molecular Probes, Eugene, Oreg.) in HBSS/BSA for 30 minutes at 37° C. Excess Calcein AM was removed with two additional washes. Normal human serum complement source (Sigma, St. Louis, Mo.) was added to a final concentration of 10, 20 or 40% in 0.05 ml volume diluted in HBSS and incubated for 1 hour at 37° C. Supernatants containing released calcein from complement lysed cells was transferred to a fresh flat bottom microtiter plate. The remaining intact cells with retained calcein were lysed with 0.05 ml 1% SDS. Released and retained fractions were read on a cytofluor 2350 (Millipore, Bedford, Mass.) at 485 nm. Data is presented as percent cell death.

FIG. 6 depicts a FACS analysis that proves that the CTLA4 domain of the chimeric molecules interacts with B7 found on the same PAECs. Co-Stimulation Assays. The costimulatory capacity of the PAEC was assayed using a modified endothelial cell costimulation assay (S. E. Maher, K. Karmann, W. Min, C. C. W. Hughes, J. S. Pober, A. L. M. Bothwell. 1996. J. Immunol. 157:3838). 5×10⁴pBABE vector control (Vector) or hCTLA4hCD59 (CC) PAECs were seeded of a 96 well plates (Becton Dickenson, Franklin Lakes, N.J.) 24 hours prior to co-culturing with T cells. The following reagents were added to final concentrations of 5 ug/ml antiCD28, or antiB7.2; 10 ug/ml antiCTLA4; or 5 ug/well sCTLA4Ig. Prior to the costimulation assay, monolayers were washed gently with DPBS three times, followed by the addition of 1×10 5 Jurkates or T cells as responder cells in 0.09 ml of RPMI 1640+FBS, and incubated for 30 min. at 37° C. Phytohemagglutinin (PHA) was added in a 0.01 ml volume to a final concentration of 10ug/ml for 20 hours at 37° C. Cell free supernatants were collected 20 hours post treatment and assayed by ELISA (R&D Systems, Minneapolis, Minn.). Plates were read on a Microplate Reader 3550 (Biorad, Hercules, Calif.) at 485 nm. Jurkat supernatants were tested undiluted.

FIG. 7 depicts the human amino acid sequence of DAF.

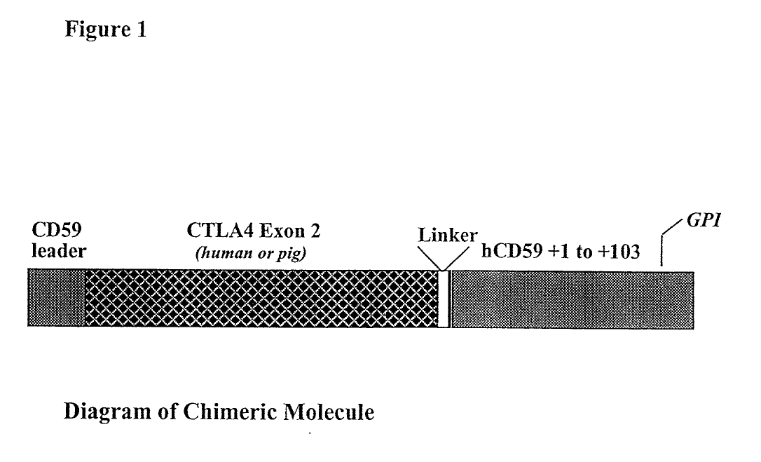

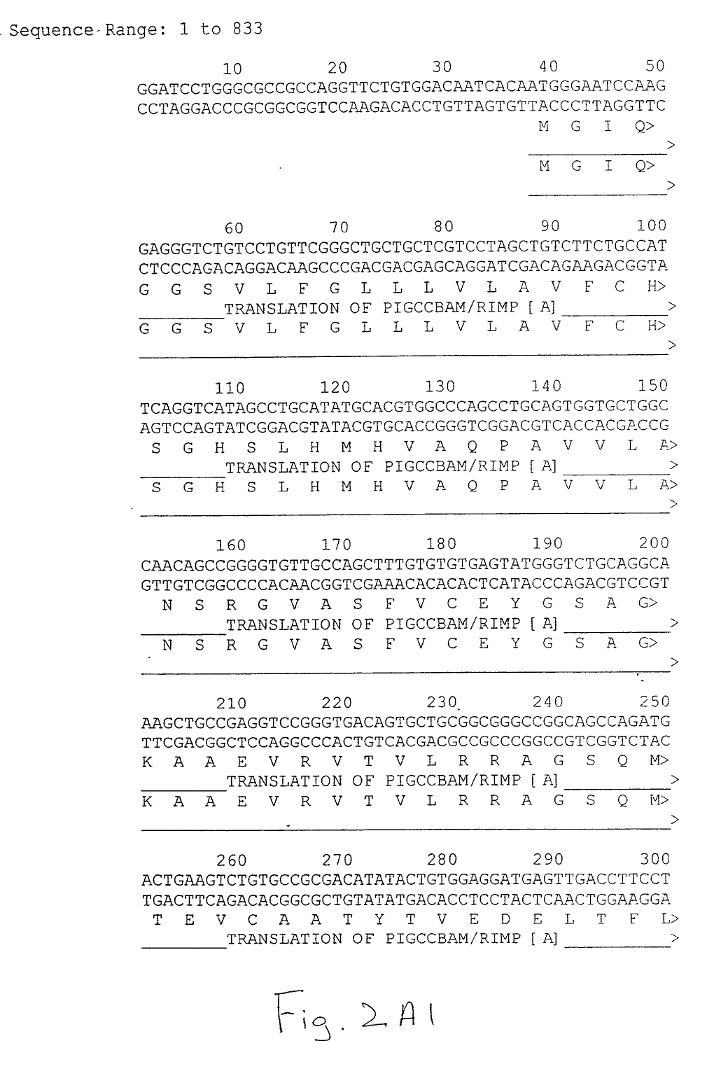

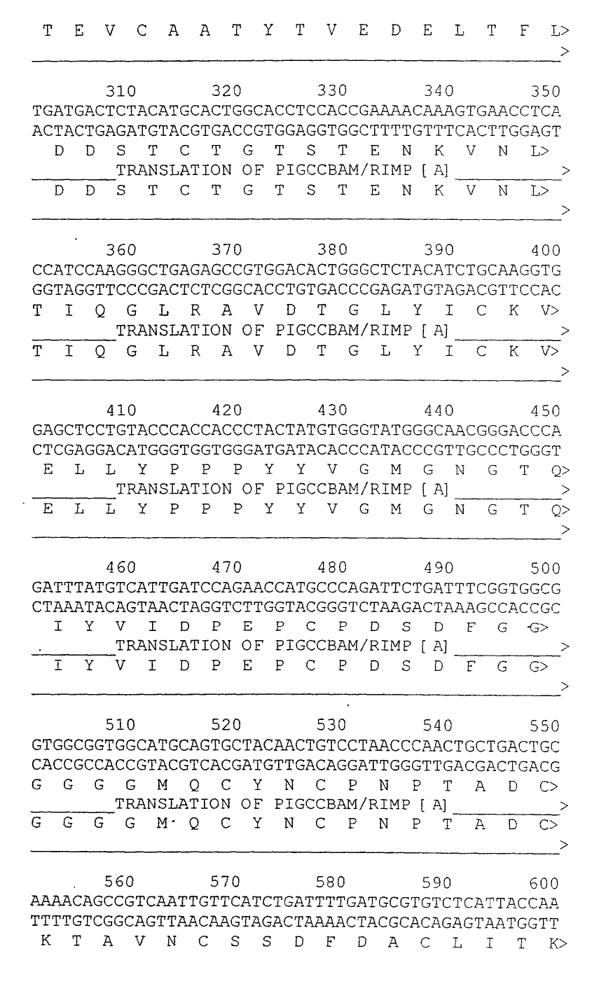

### DETAILED DESCRIPTION

It has been found that functional domains capable of regulating the humoral effector functions of the immune system including complement inhibitory domains, such as a C5b-9 inhibitory domains or C3 inhibitory domains; and functional domains capable of regulating the cellular effector functions of the immune system, including T Cell inhibitory domains, can advantageously be combined to form a chimeric protein. The chimeric protein can be expressed on a porcine cell surface and can aid in the protection of the porcine cell, after xenotransplantation into a human, from both the human cellular immune response and human complement.

As used herein, the phrase “C5b-9 inhibitory activity” is used herein to describe the effects of C5b-9 inhibitor molecules of the foregoing types on the complement system and thus includes activities that lead to inhibition of the cell activating and/or lytic function of the membrane attack complex (MAC).

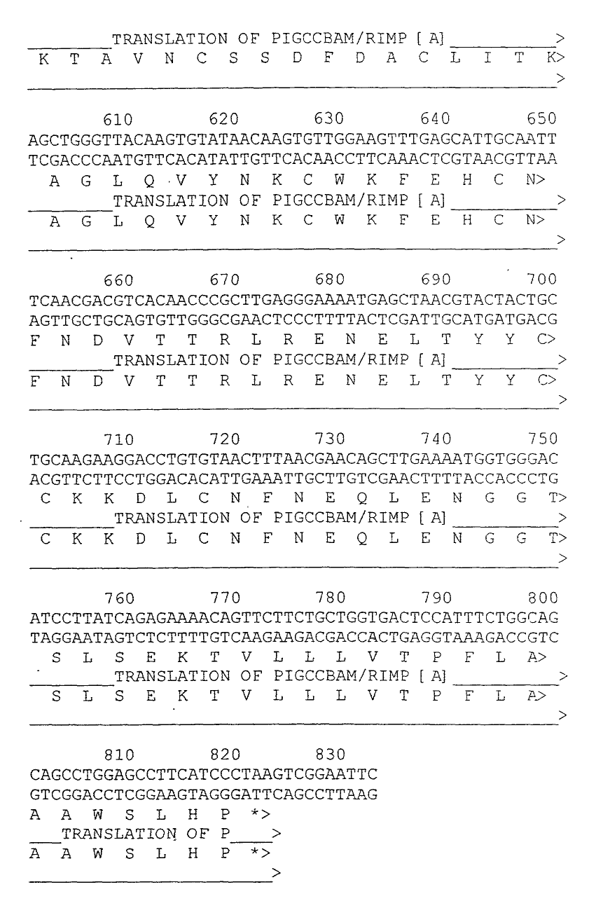

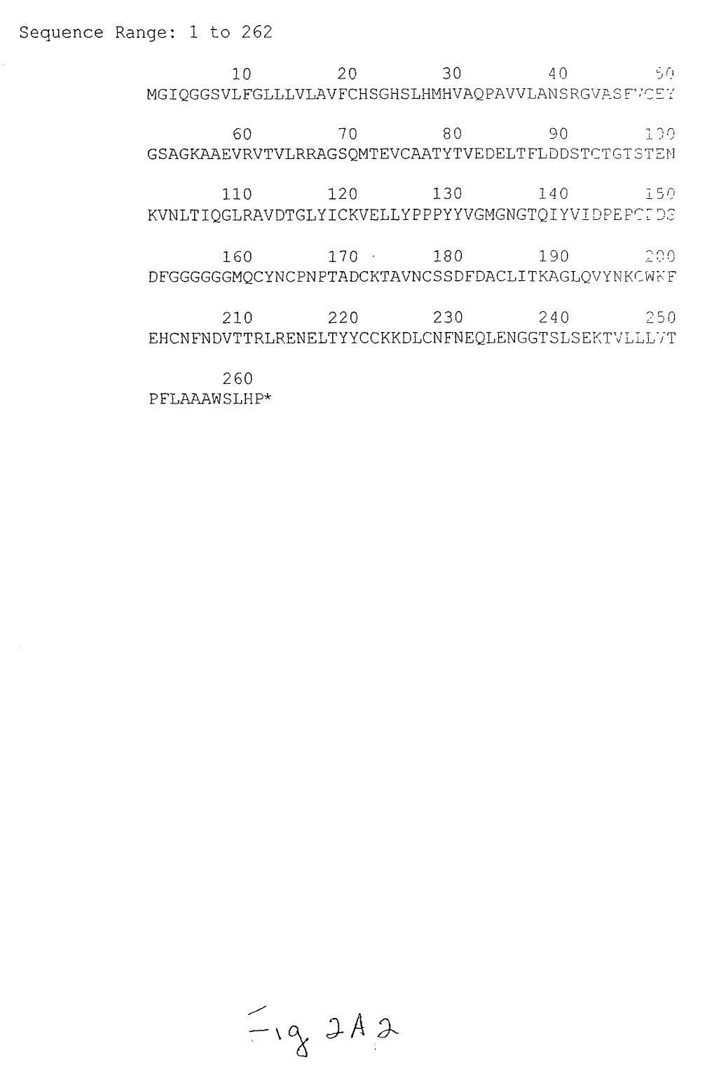

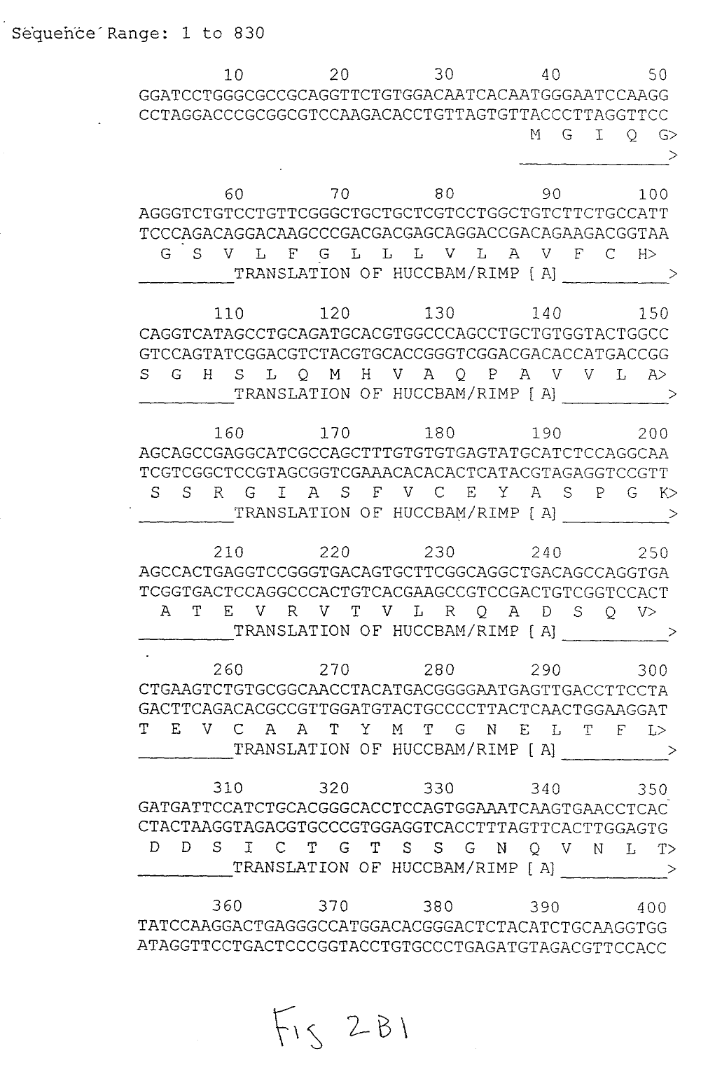

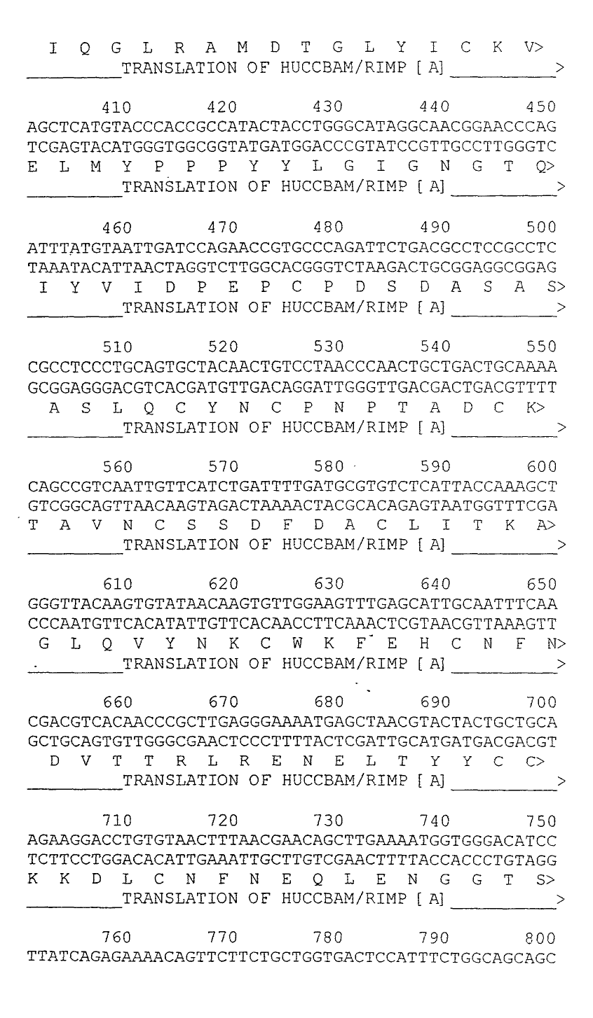

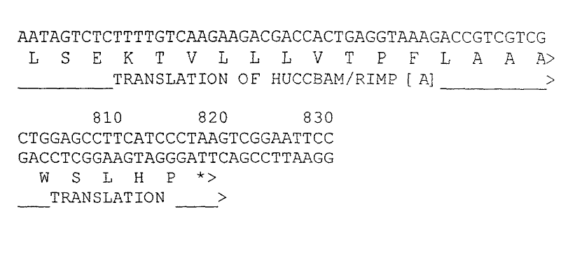

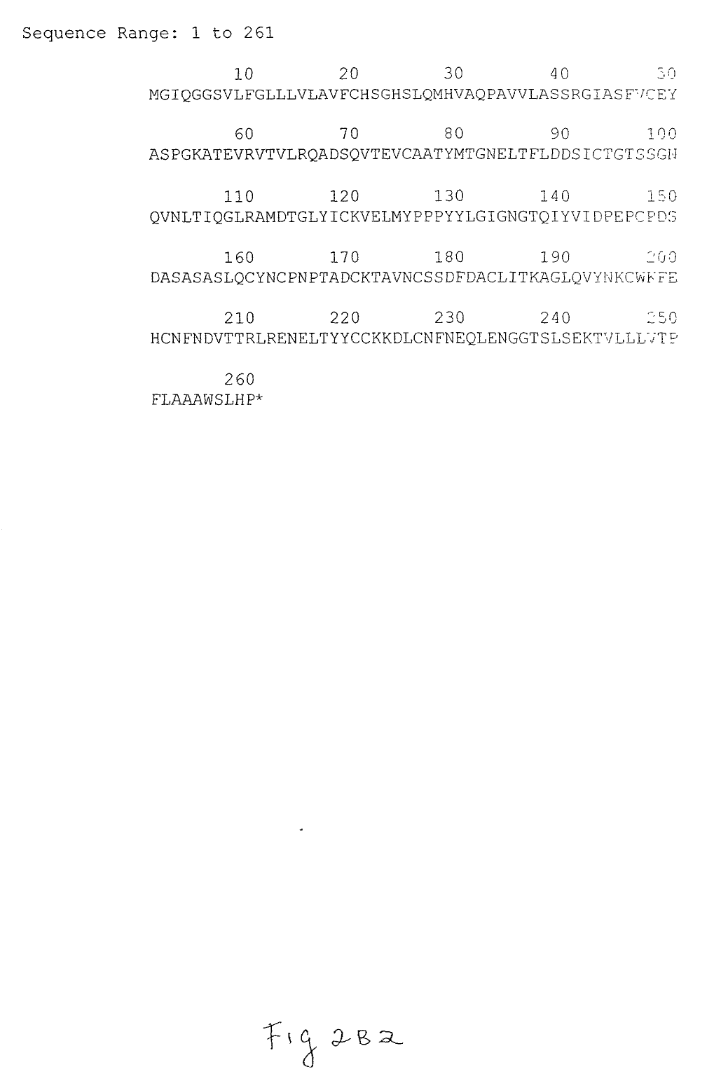

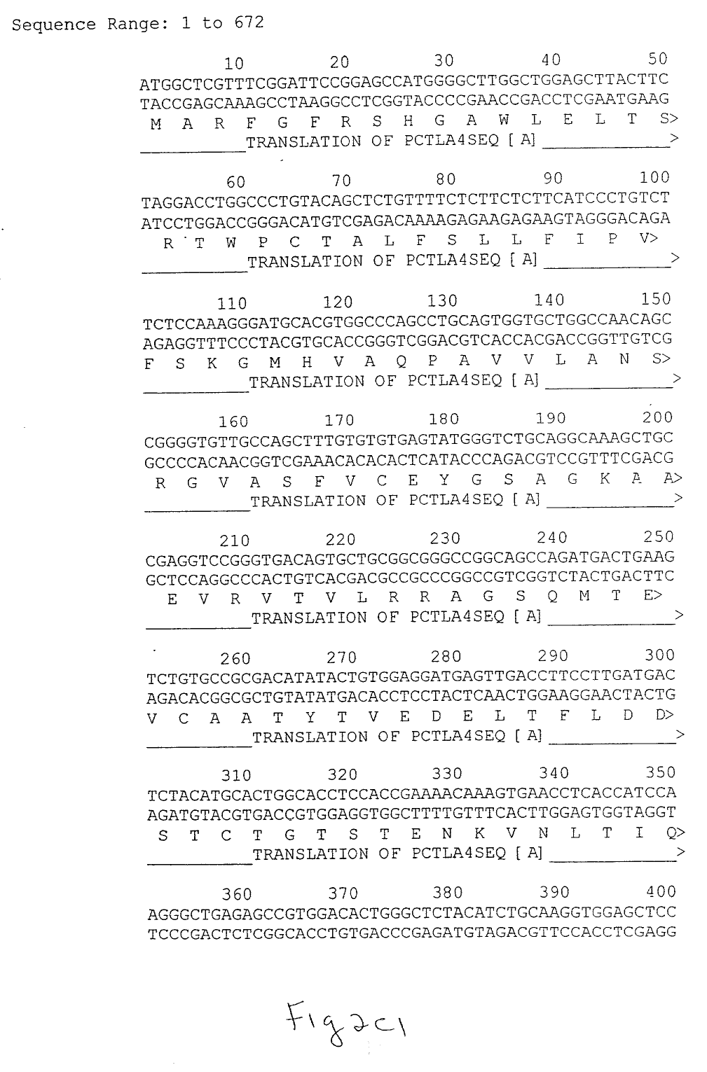

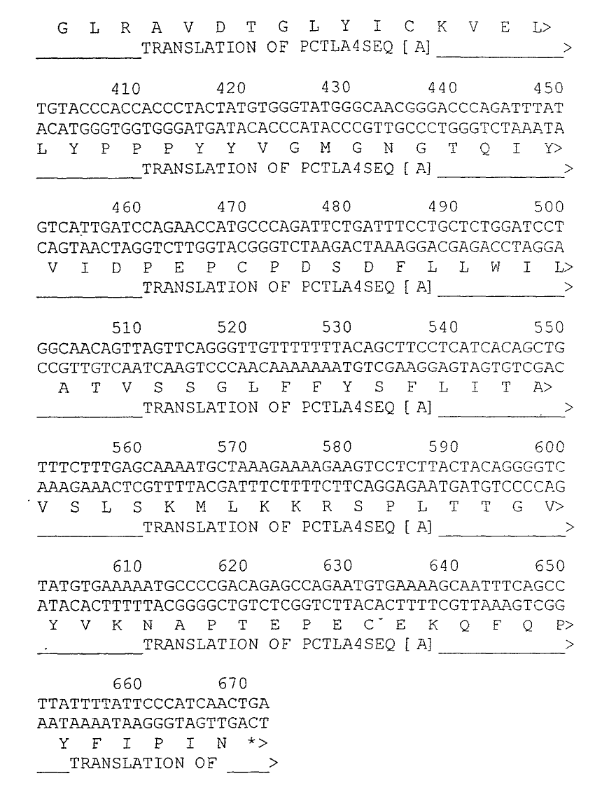

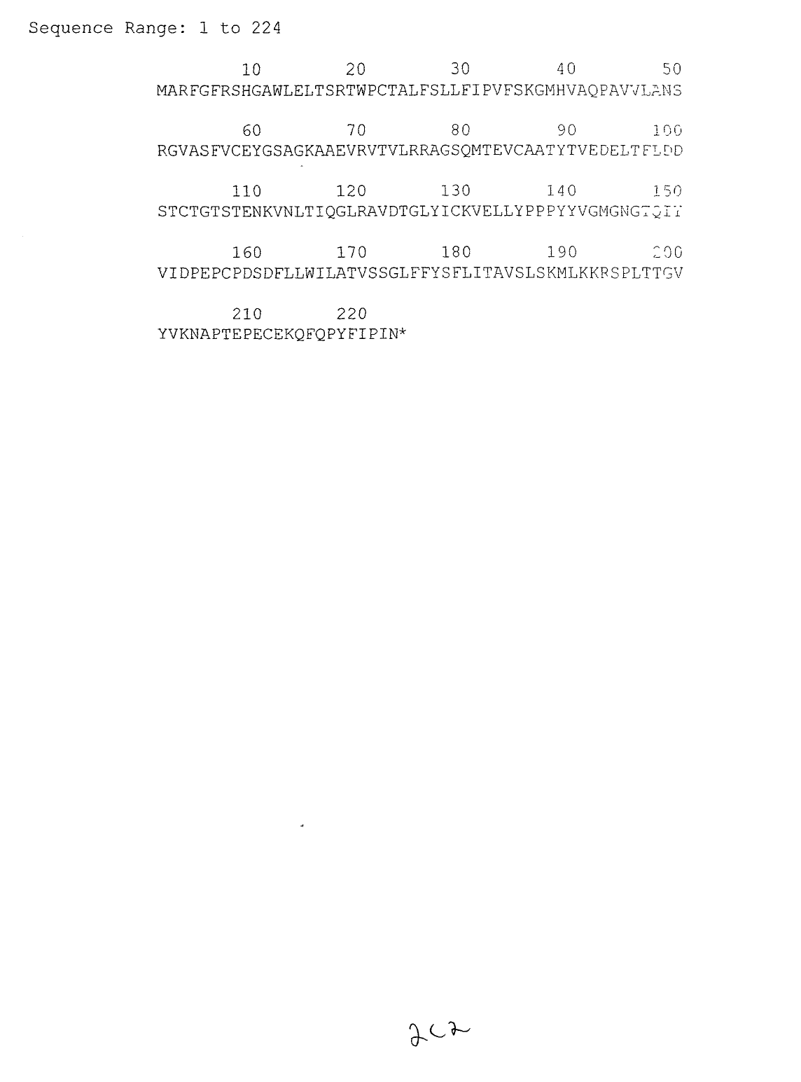

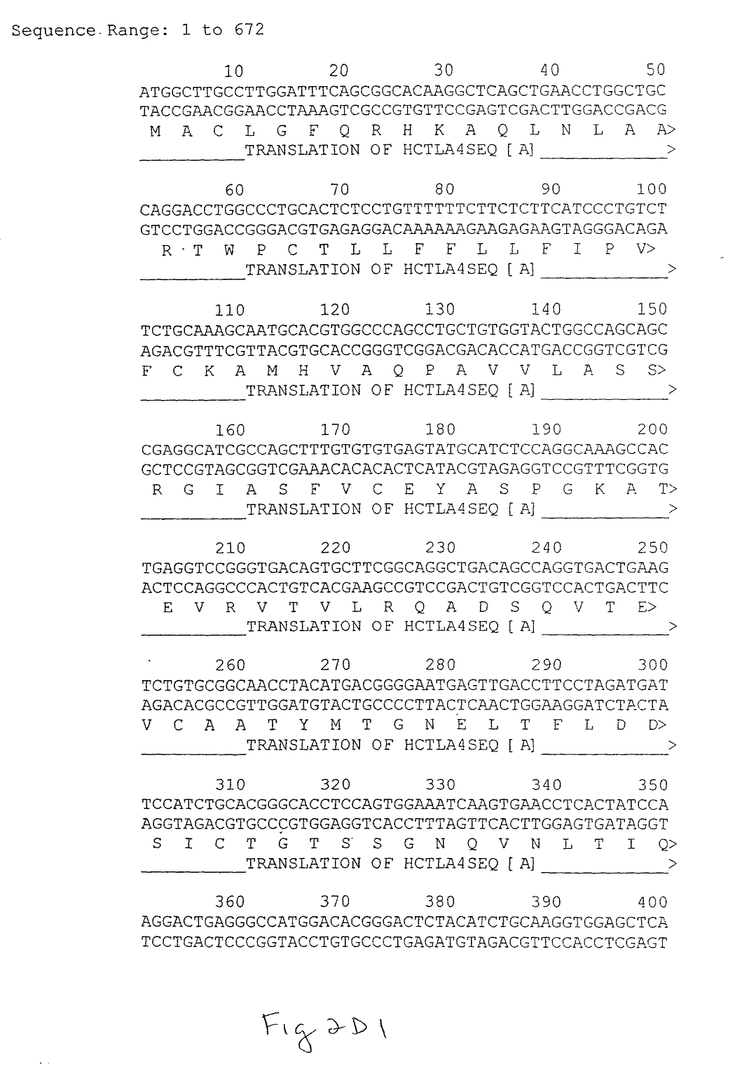

Suitable domains which exhibit C5b-9 inhibitory activity can include the entire amino acid sequence for a naturally occurring C5b-9 inhibitor protein or a portion thereof. For example, the C5b-9 sequence can be the mature CD59 molecule (i.e. amino acids 1 through 103 of FIG. 2E(2)). Alternatively, the C5b-9 sequence can be a portion of a naturally occurring C5b-9 inhibitor protein, such as CD59. Active portions suitable for use herein can be identified using a variety of assays for C5b-9 inhibitory activity known in the art. See for example Rollins, et al., J. Immunol. 144:3478, 1990; Rollins, et al., J. Immunol. 146:2345, 1991; Zhao, et al., J. Biol. Chem. 266:13418, 1991; and Rother, et al., J. Virol. 68:730, 1994. In general, the portion used should have at least about 25% and preferably at least about 50% of the activity of the parent molecule.

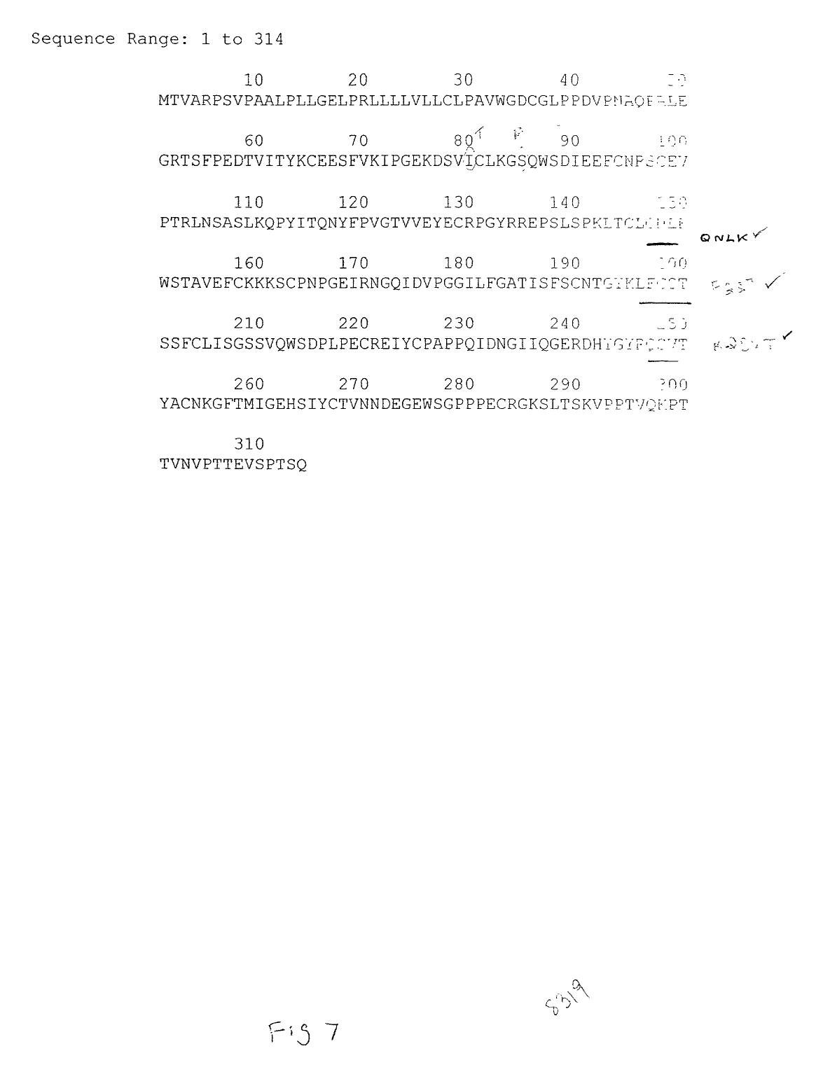

Suitable C3 inhibitory domains include the entire amino acid sequence for a naturally occurring C3 inhibitor or a portion thereof, such as one or more SCRs of the C3 inhibitory domain. For example, the C3 sequence can be the mature DAF molecule (factor H, membrane cofactor protein or complement receptor 1). Alternatively, the C3 inhibitory domain can be a portion of a naturally occurring C3 inhibitor protein. Following the procedures used to identify functional domains of DAF (Adams, et al., 1991. J. Immunol. 147:3005-3011), functional domains of other C3 inhibitors can be identified and used herein. In general, the portion used should have at least about 25% and preferably at least about 50% of the activity of the parent C3 inhibitory molecule. Particularly useful portions of mature C3 inhibitor proteins include one or more of the mature molecule's SCRs. These SCRs are normally approximately 60 amino acids in length and have four conserved cysteine residues which form disulfide bonds, as well as conserved tryptophan, glycine, and phenylalanine/tyrosine residues. In one embodiment the C3 inhibitory domain includes SCRs 2 through 4 of DAF (i.e. amino acids 97 through 286 shown in FIG. 7).

Suitable domains which exhibits T Cell inhibitory activity can include either at least a portion of the amino acid sequence for naturally occurring porcine CTLA4 or at least a portion of the entire amino acid sequence for naturally occurring human CTLA4. For example, the amino acid sequence which exhibits T Cell inhibitory activity can be amino acids 38 to 162 of the porcine CTLA4 sequence shown in FIG. 2C(2) or amino acids 38 to 161 of the human CTLA4 sequence shown in FIG. 2D(2). In general, the portion used should have at least about 25% and preferably at least about 50% of the activity of the parent molecule.

The amino acid sequence having C5b-9 inhibitory activity and the amino acid sequence having T Cell inhibitory activity do not have to be directly attached to one another. A linker sequence can separate these two sequences. The linker preferably comprises between about one and at least about 6 amino. Suitable linker sequences can include glycines. Other amino acids, as well as combinations of amino acids, can be used in the linker region if desired. In one embodiment, amino acids 153 to 158 of FIG. 2A(2) (GGGGGG in pCC) are the linker sequence. In another embodiment, amino acids 152 to 157 of FIG. 2B(2) (SASASA in hCC) are the linker sequence.

Another embodiment provides recombinant cDNA that encodes an exon of the human homologue of CTLA4 is inserted into the coding region of human CD59, bisecting CD59 between the leader peptide and the mature peptide post-translational processing site, see FIG. 2A(1). A further embodiment provides recombinant cDNA which that encodes an exon of the porcine homologue of CTLA4 is inserted into the coding region of human CD59, bisecting CD59 between the leader peptide and the mature peptide post-translational processing site, see FIG. 2B(1). In both embodiments, the cDNA may include a coding sequence for a GPI anchor linkage site corresponding to amino acid 210 of CC and amino acid 77 of native CD59, see FIGS 2A(1) and 2B(1).

Molecules comprising nucleotide sequences encoding the CTLA4 and CD59 or DAF domains can be prepared using a variety of techniques known in the art. For example, the nucleotide sequences encoding the CTLA4 nucleotide # 112-483 and CD59 leader peptide region nucleotide 1-75 and mature peptide nucleotide 76-384 domains can be produced using PCR generation and/or restriction digestion of cloned genes to generate fragments encoding amino acid sequences having T Cell and C5b-9 inhibitory activities. These fragments can be assembled using PCR fusion or enzymatic ligation of the restriction digestion products (Sambrook, et al., Molecular Cloning: A laboratory manual. 2^{nd} edition. Cold Spring Harbor Press, 1989); Ausubel, et al. Current Protocols in Molecular Biology. 1991) Alternatively, any or all of the nucleic acid fragments used to assemble the chimeric genes can be synthesized by chemical means. In another embodiment, the nucleotide sequences encoding the CTLA4 and DA1 domains can be produced using PCT generation and/or restriction digestion of cloned genes to generate fragments encoding amino acid sequences having T Cell and C3 inhibitory activities. These fragments also can be assembled using PCR fusion or enzymatic ligation of the restriction digestion products (Sambrook, et al., Molecular Cloning: A laboratory manual. 2^{nd} edition. Cold Spring Harbor Press, 1989; Ausubel, et al., Current Protocols in Molecular Biology. 1991). Any or all of the nucleic acid fragments used to assemble these chimeric genes can be synthesized by chemical means as well.

In another embodiment, recombinant expression vectors which include nucleic acid fragments the chimeric protein are provided. The nucleic acid molecule coding for such a chimeric protein can be inserted into an appropriate expression vector, i.e., a vector that contains the necessary element for the transcription and translation of the inserted protein-encoding sequence. Suitable host vector systems include, but are not limited to, mammalian cell systems infected with virus (e.g., vaccinia virus, adenovirus, retroviruses, etc.); mammalian cell systems transfected with plasmids; insect cell systems infected with virus (e.g., baculovirus); microorganisms such as yeast containing yeast expression vectors, or bacteria transformed with bacteriophage DNA, plasmid DNA, or cosmid DNA (see, for example, Goeddel, 1990). Commonly used promoters and enhancers derived from Polyoma virus, Adenovirus, Simian Virus 40 (SV40), the Molony murine leukemia virus (MMLV), including the long terminal repeat (MMLVLTR), and human cytomegalovirus (CMV), including the cytomegalovirus immediate-early gene 1 promoter and enhancer are suitable. Eukaryotic promotors-BetaActin (NG et al) & H2 Kb (Fodor et al. PNAS 1994).

In a preferred embodiment, the cDNA of interest is cloned into a retroviral vector that is subsequently transfected into a mouse cell line called a “packaging line”. The manipulation of retroviral nucleic acids to construct retroviral vectors and packaging cells is accomplished using techniques known in the art. See for example, Ausubel, et al., 1992, Volume 1, Section III (units 9.10.1-9.14.3); Sambrook, et al., Molecular Cloning: A laboratory manual. 2^{nd} edition. Cold Spring Harbor Press, 1989; Miller, et al., Molecular and Cellular Biology 6:2895, 1986; Eglitis, et al., Biotechniques. 6:608-614. 1988; U.S. Pat. Nos. 4,650,764, 4,861,719, 4,980,289, 5,122,767, and 5,124,263; as well as PCT Patent Publications Nos. WO 85/05629, WO 89/07150, WO 90/02797, WO 90/02806, WO 90/13641, WO 92/05266, WO 92/07943, WO 92/14829, and WO 93/14188. Typically, the retroviral vector contains a gene that allows for selection via resistance to drugs such as puromyacin. It also contains nucleic acid sequence that allows for random or directed integration of the vector into a eukaryotic genome. Drug resistant cell lines are selected. These cells will produce virus particles capable of infecting other cells lines. Porcine aortic endothelia cells (PAECs) are infected with the viruses by a process called viral transduction. The transduced PAECs are selected for by drug resistance. Drug resistant cells contain integrated copy of the viral vector DNA. Once in the porcine genome, vector sequences or sequences associated with the chimeric gene control the expression of the chimeric protein.

In particular, the retroviral vectors of the invention can be prepared and used as follows. First, a retroviral vector containing nucleic acid encoding for the chimeric protein described herein above is constructed and packaged into non-infectious transducing viral particles (virions) using an amphotropic packaging system, preferably one suitable for use in gene therapy applications. Examples of such packaging systems are found in, for example, Miller, et al., Molecular and Cellular Biology 6:2895, 1986; Markowitz, et al., J. Virol. 62:1120-1124. 1988; Cosset, et al., J. Virol. 64:1070-1078. 1999. U.S. Pat. Nos. 4,650,764, 4,861,719, 4,980,289, 5,122,767, and 5,124,263, and PCT Patent Publication Nos. WO 85/05629, WO 89/07150, WO 90/02797, WO 90/02806, WO 90/13641, WO 92/05266, WO 92/07943, WO 92/14829, and WO 93/14188. A preferred packaging cell is the PA317 packaging cell line (ATCC CRL 9078, Rockville, Md.). The generation of “producer cells” is accomplished by introducing retroviral vectors into the packaging cells. The producer cells generated by the foregoing procedures are used to produce the retroviral vector particles (virions). This is accomplished by culturing of the cells in a suitable growth medium. Preferably, the virions are harvested from the culture and administered to the target cells which are to be transduced. Examples of such retroviral vectors are found in, for example, Korman, et al., Proc. Natl. Acad. Sci. USA. 84:2150-2154. 1987; Morgenstern, et al., Nucleic Acid Research 18:3587. 1990; U.S. Pat. Nos. 4,405,712, 4,980,289, and 5,112,767; and PCT Patent Publication Nos. WO 85/05629, WO 90/02797, and WO 92/07943. A preferred retroviral vector is the MMLV derived expression vector pLXSN (See Miller, et al., Biotechniques. 7:981, 1989). DNA can be introduced into cells by any standard method of transfection such as polybrene, DEAE, calcium phosphate, lipofection, electroporation. (See Sambrook, et al., Molecular cloning: a laboratory manual. Second Edition. Cold Spring Harbor Laboratory Press, Cold Spring Harbor, N.Y. 1989).

Engineered transgenic animals (for example, rodent, e.g., mouse, rat capybara, and the like, lagomorph, e.g., rabbit, hare, and the like, ungulate, e.g., pig, cow, goat, sheep, and the like, etc.) that express the chimeric protein described herein on the surfaces of their cells are provided using any suitable techniques known in the art. These techniques include, but are not limited to, microinjection, e.g., of pronuclei, electroporation of ova or zygotes, nuclear transplantation, and/or the stable transfection or transduction of embryonic stem cells derived from the animal of choice.

A common element of these techniques involves the preparation of a transgene transcription unit. Such a unit includes a DNA molecule which generally includes: 1) a promoter, 2) the nucleic acid sequence, and 3) a polyadenylation signal sequence. Other sequences, such as, enhancer and intron sequences, can optionally be included. The unit can be conveniently prepared by isolating a restriction fragment of a plasmid vector which expresses the CTLA4-CD59 protein in, for example, mammalian cells. Preferably, the restriction fragment is free of bacterially derived sequences that are known to have deleterious effects on embryo viability and gene expression.

The most well known method for making transgenic animals is that used to produce transgenic mice by superovulation of a donor female, surgical removal of the egg, injection of the transgene transcription unit into the pro-nuclei of the embryo, and introduction of the transgenic embryo into the reproductive tract of a pseudopregnant host mother, usually of the same species. See for example U.S. Pat. No. 4,873,191, Brinster, et al., 1985. Proc. Natl. Acad. Sci. USA. 82:4438-4442., Hogan, et al., in “Manipulating the Mouse Embryo: A Laboratory Manual”. Cold Spring Harbor Laboratory, Cold Spring Harbor, N.Y., 1986., Robertson. 1987. In Robertson. Ed. “Teratocarcinomas and Embryonic Stem Cells a Practical Approach” IRL Press, Eynsham, Oxford, England, Pedersen, et al., 1990. “Transgenic Techniques in Mice—A Video Guide”, Cold Spring Harbor Laboratory, Cold Spring Harbor, N.Y.

The use of this method to make transgenic livestock is also widely practiced by those of skill in the art. As an example, transgenic swine are routinely produced by the microinjection of a transgene transcription unit into pig embryos. See, for example, PCT Publication No. WO 92/11757. In brief, this procedure may, for example, be preformed as follows. First, the transgene transcription unit is gel isolated and extensively purified through, for example, an ELUTIP column (Schleicher & Schuell, Keene, N.H.), dialyzed against pyrogen free injection buffer (10 mM Tris, pH 7.4+0.1 mM EDTA in pyrogen free water) and used for embryo injection. Embryos are recovered from the oviduct of a hormonally synchronized, ovulation induced sow, preferably at the pronuclear stage. They are placed into a 1.5 ml microfuge tube containing approximately 0.5 ml of embryo transfer media (phosphate buffered saline with 10% fetal calf serum). These are centrifuged for 12 minutes at 16,000.times.g in a microcentrifuge. Embryos are removed from the microfuge tube with a drawn and polished Pasteur pipette and placed into a 35 mm petri dish for examination. If the cytoplasm is still opaque with lipid such that the pronuclei are not clearly visible, the embryos are centrifuged again for an additional 15 minutes. Embryos to be microinjected are placed into a drop of media (approximately 100.mu.1) in the center of the lid of a 100 mm petri dish. Silicone oil is used to cover this drop and to fill the lid to prevent the medium from evaporating. The petri dish lid containing the embryos is set onto an inverted microscope equipped with both a heated stage (37.5.degree.-38.degree. C.) and Hoffman modulation contrast optics (200.times. final magnification). A finely drawn and polished micropipette is used to stabilize the embryos while about 1-2 picoliters of injection buffer containing approximately 200-500 copies of the purified transgene transcription unit is delivered into the nucleus, preferably the male pronucleus, with another finely drawn and polished micropipette. Embryos surviving the microinjection process as judged by morphological observation are loaded into a polypropylene tube (2 mm ID) for transfer into the recipient pseudopregnant sow. Offspring are tested for the presence of the transgene by isolating genomic DNA from tissue removed from the tail of each piglet and subjecting about 5 micrograms of this genomic DNA to nucleic acid hybridization analysis with a transgene specific probe. In a preferred embodiment, transgenic animals are produced according to the methods disclosed in PCT Publication No. WO 99/07829, the contents of which are incorporated herein by reference.

Another commonly used technique for generating transgenic animals involves the genetic manipulation of embryonic stem cells (ES cells) as described in PCT Publication No. WO 93/02188 and Robertson, in Robertson. ed. “Teratocarcinomas and Embryonic Stem Cells a Practical Approach” IRL Press, Eynsham, Oxford, England 1987. In accordance with this technique, ES cells are grown as described in, for example, Robertson, in Robertson, ed. “Teratocarcinoma and Embryonic Stem Cells a Practical Approach” IRL Press, Eynsham, Oxford, England; 1987, and in US. Patent No. 5,166,065 to Williams et al. Genetic material is introduced into the embryonic stem cells by, for example, electroporation according, for example, to the method of McMahon, et al., Cell. 62:1073; 1990, or by transduction with a retroviral vector according, for example, to the method of Robertson, et al., Nature. 323:445; 1986, orby any of the various techniques described by in Robertson, ed. “Teratocarcinomas and Embryonic Stem Cells a Practical Approach” IRL Press, Eynsham, Oxford, England, 1987. Chimeric animals are generated as described, for example, in Bradley, in Robertson. ed. “Teratocarcinomas and Embryonic Stem Cells a Practical Approach” IRL Press, Eynsham, Oxford, England, 1,987. Briefly, genetically modified ES cells are introduced into blastocysts and the modified blastocysts are then implanted in pseudo-pregnant female animals. Chimeras are selected from the offspring, for example by the observation of mosaic coat coloration resulting from differences in the strain used to prepare the ES cells and the strain used to prepare the blastocysts, and are bred to produce non-chimeric transgenic animals.

Other methods for the production of transgenic animals are disclosed in U.S. Pat. No. 5,032,407 and PCT Publication No. WO 90/08832.

In order that those skilled in the art may be better able to practice the compositions and methods described herein, the following examples are given an illustration of the preparation of chimeric proteins having both complement inhibitory domains and T Cell inhibitory domains, as well as their ability to be expressed on a cell surface of an antigen presenting cell, bind targets on the same antigen presenting cell, and exhibit T Cell inhibitory activity. It is to be understood that commercially available reagents and/or instrumentation referred to in the examples were used according to the manufacturer's instruction unless otherwise indicated.

### EXAMPLE 1

Construction of Human CTLA4-Human CD59 Chimeric Molecules

A full length human CTLA4 cDNA was isolated from human peripheral blood leukocytes (PBLs) that were activated with 3 ng/ml phorbol 12 myristate 13 acetate (PMA) and 0.4 ug/ml ionomycin (commercially available from Sigma, St. Louis, Mo.). First strand CDNA synthesized from PBL RNA using reverse transcriptase as recommended by the vendor (Seikagaku America, Inc., Rockville, Md.) was used as a template in a polymerase chain reaction (PCR) to amplify the extracellular domain encoded by exon 2 (according to methods described in Brunet, et al, “A Differential Molecular Biology Search for Genes Preferentially Expressed in Functional T Lymphocytes: The CTLA Genes”, Immunol. Rev. 103:21-36 (1988), and Dariavach, et al, Human Ig Superfamily CTLA-4 Gene: Chromosomal Localization and Identity of Protein Sequence Between Murine and Human CTLA-4 Cytoplasmic Domains,: Eur. J. Immunol. 18:1901-1905 (1988).). The 5′ forward oligonucleotide: 5′GCCTGCAGATGCACGTGGCC3′ and the 3′ reverse oligonucleotide; 5′GGCTGCAGGGAGGCGGAGGCGGAGGCGTCAGAATCTGG3′, which contained homologous nucleotides and nucleotide encoding linker sequence, were used in the following PCR reaction mixture to amplify a 406 base pair CTLA4 DNA fragment. Five microliters of a first strand synthesis of cDNA made from activated PBLs was amplified in the presence of 10 mM magnesium chloride, 500 mM dNTPs, 2 uM oligonucleotides, 2.5 Units Taq polymerase (Perkin Elmer, Norwalk, Conn.) for forty cycles. Each cycle consisted of denaturing for one minute at 95° C., annealing at 55° C. for one minute, and polymerizing at 72° C. for one minute. One cycle of polymerization at 72° C. for ten minutes insured the addition of thymidine overhand for TA cloning. The CTLA4 exon 2 fragment was ligated into the pCRII. 1 TOPO vector using the TOPO TA cloning kit (commercially available from Invitrogen, Carlsbad, Calif.) and used to transform the TOP 10 strain of E. Coli (commercially available from Invitrogen, Carlsbad, Calif.). Plasmids containing the appropriately sized fragment were isolated and the inserts were subjected to DNA sequencing to confirm the integrity and identity of the DNA (Wm. Keck Foundation Biotechnology Resource Laboratory Yale University, New Haven, Conn.). Plasmids that contained the verified CTLA4 exon 2 insert were digested with PstI and the 406 bp CTLA4 exon 2 fragment was isolated. A GEM7Z plasmid (commercially available from Clontech, Palo Alto, Calif.) that contained the human CD59 sequence (gift from Dr. A. Bothwell, Yale University) that has a unique PstI site located between the human CD59 signal sequence and mature protein coding sequence was digested with PstI. The PstI fragment that contained CTLA4 exon 2 was ligated into the corresponding PstI site on GEM7Z and plasmids that contained the correct insert were selected. A BamHI-EcoRI fragment containing the entire chimeric human CTLA-human CD59 (hCTLA4hCD59) gene was excised from the plasmid and then subcloned into the amphotropic retroviral expression vector pBABEpuro (Morganstem, et al) to generate the expression vector hCTLA4hCD59BABEpuro.

### EXAMPLE II

Cloning of Porcine CTLA4

Porcine cDNA was prepared from porcine PBLs that were activated with 3 ng/ml PMA and 0.4 ug/ml ionomycin (Commercially available from Sigma, ST. Louis, Mo.). The cDNA was used as a template in a PCR using redundant primers designed from a comparison of human (Genbank accession # NM005214), mouse (X05719), rabbit (D49844), and bovine (X93305) CTLA4 nucleotide sequences. The 5′ foreward oligonucleotide: 5′CCCMYMAGCCATGGCTYYYGG3′ together with the 3′ reverse oligonucleotide: 5′CCTCARTTRATRGGA4AAAATAAGGTG3′ were used in PCR conditions as described in Example I, except annealing occurred at 45C, and twenty cycles of amplification were used. The PCR produced a 672 base pair fragment that was cloned into the TOPO vector using the TOPO TA cloning kit (commercially available from Invitrogen, Carlsbad, Calif.). DNA sequence analysis confirmed that the insert was a full-length porcine CLTA4 clone.

### EXAMPLE III

Construction of Porcine CTLA4-Human CD59 Chimeric Molecules

The extracellular domain of porcine CTLA4 encoded by exon 2 was PCR amplified from the TOPO plasmid prepared in Example II using a 5′ foreward oligonucleotide: 5′CCATGCATAT GCACGTGGCC CAGCCTGCAG, and a 3′ oligonucleotide: 5′CATGCATGCC ACCGCCACC GCCACCGAAA TCAGAATCTG GGCATGGTTC TGGATCAATG3′ that contained homologous pCTLA4 sequence restriction sites and linker sequence using the same PCR conditions as described in Example I, except that only 35 cycles of amplification were used to generate a 393 base pair DNA fragment. The fragment was cloned into the TOPO vector using the TOPO TA cloning kit (commercially available from Invitrogen, Carlsbad, Calif.). DNA sequence analysis confirmed that the insert was the porcine CTLA4 exon 2. The plasmid was digested with Nsi I and the fragment that contained the CTLA4 exon 2 was isolated and ligated into the PstI site of the GEM7Z plasmid that contained human CD59 as described in Example I. The BamE1-EcoR1 fragment containing the chimeric pig CTLA4-human CD59 molecule (pCTLA4hCD59) was excised from the plasmid and then subcloned into the amphotropic retroviral expression vector pBABEpuro (See Morgenstern, et al., Nucleic Acids Res. 18:3587 1990.) to generate the expression vector pCTLA4hCD59BABEpuro.

### EXAMPLE IV

Cell Surface Expression of hCTLA4-hCD59 Chimeric Molecules

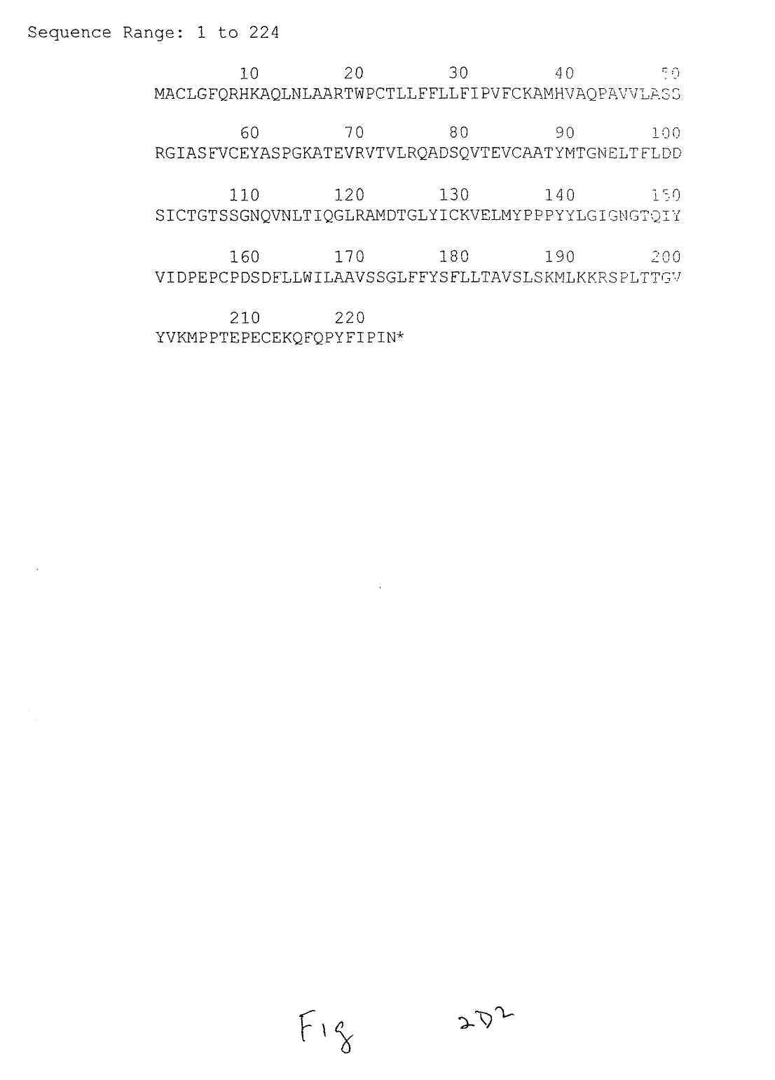

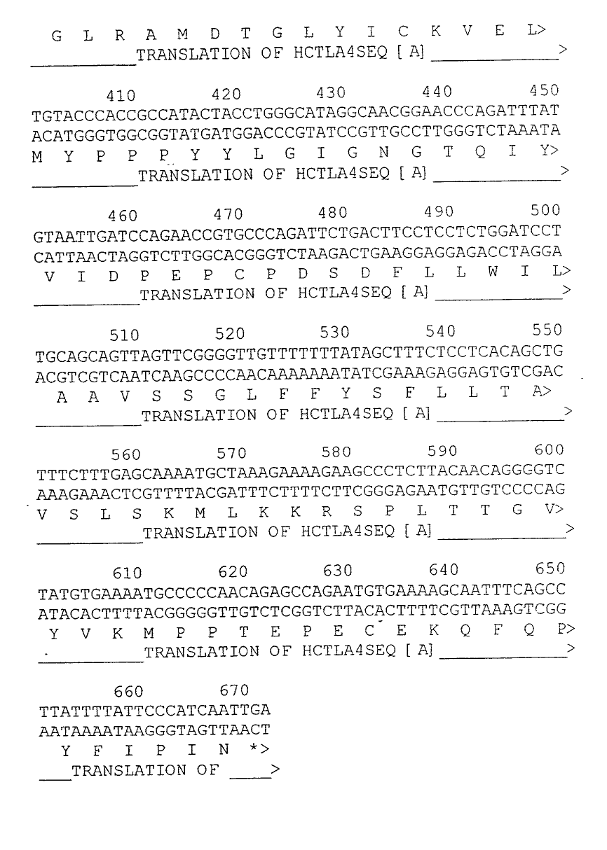

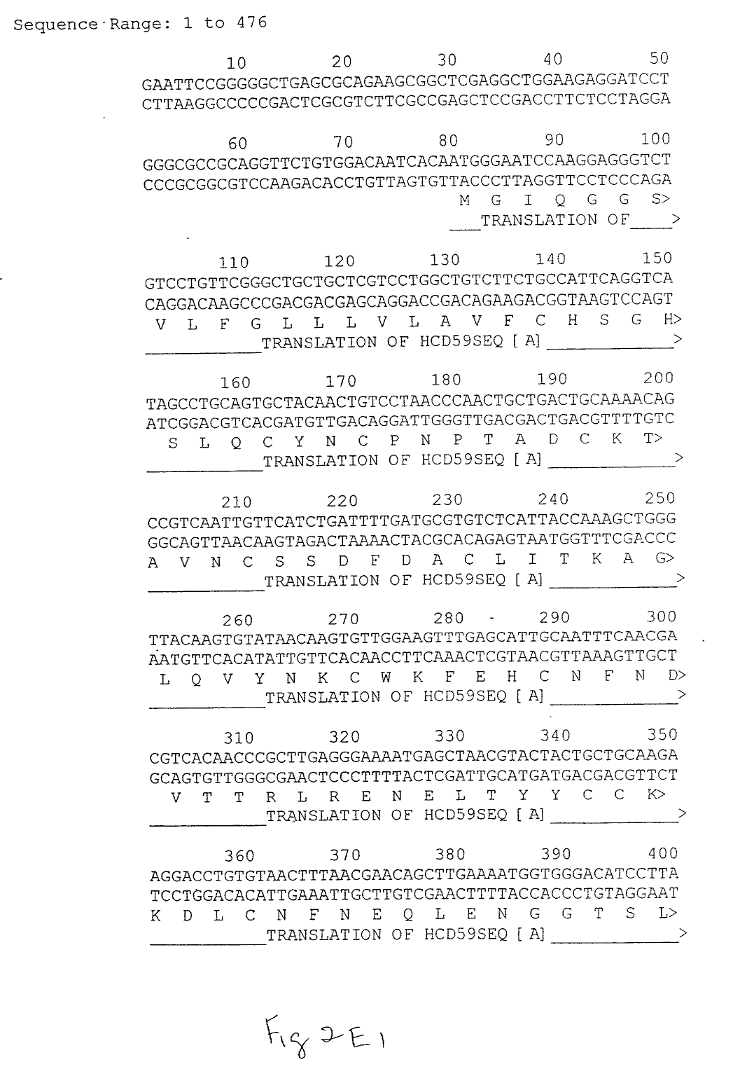

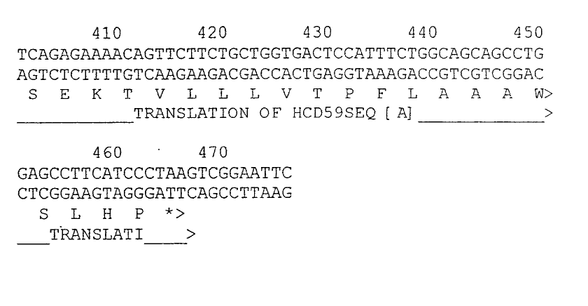

To create a cell line that expresses the human CTLA4-humanCD59 chimeric molecule a cell line must be created that produces retroviral vectors that contain the necessary gene. Another cell line must then be transduced with the virus to create a population of cells that express the protein. To produce the retrovirus, the murine amphotropic packaging cell line PA317 (ATCC, Rockville, Md.) was transfected with the expression vector prepared in Example I (hCTLA4hCD59BABEpuro), example 3 or BABEpuro (vector control DNA) by the polybrene method (See Sambrook, et al., Molecular cloning: a laboratory manual. Second Edition. Cold Spring Harbor Laboratory Press, Cold Spring Harbor, N.Y. 1989) Ten micrograms of DNA were added to PA317 cells in 5 ml of Dulbecco minimum essential medium (DMEM), available from Cellgro, Herndon, Va. containing 10% heat inactivated fetal bovine serum (FBS) followed by a five hour treatment with 30 mg/ml polybrene (Sigma, St. Louis, Mo.), without a dimethylsufoxide (DMSO) shock. The cells were washed and incubated in DMEM with 10% FBS and 48 hours post transfection, the cells were treated with 3 mg/ml puromycin to select drug resistant transfectants. The transfected PA317 cells produced retrovirus and viral supernatants which were harvested as described by Morgenstern, et al, Land, H. Advanced mammalian gene transfer: high titre retroviral vectors with multiple drug selection markers and a complementary helper free packaging cell line. 1990. Nucleic Acid Research. 18:3587. The next step was to transduce porcine cells to create a porcine cell line that expresses human CTLA4-humanCD59 protein and the porcine CTLA4-hCD59. Using standard methods 5×105 porcine aortic endothelial cells (PAEC) were transduced using 1.5 ml of viral supernatant added to 3.5 ml of DMEM with 10% FBS followed by the addition of polybrene to 8 mg/ml for 5 hours. Following transduction the cells were incubated in DMEM with 10% FBS for 48 hours. The cells were split into selection medium (DMEM with 10% FBS and 3 ug/ml puromycin). Puromycin resistant cell populations that express human CTLA4-human CD59 chimeric molecules were identified by florescence activated cell sorting (FACS) analysis using antibodies to human CTLA4 and human CD59 using standard methodologies (Current Protocols in Immunology, ed. J. E. Coligan, et al). Populations of PAEC transduced with hCTLA4hCD59BABEpuro, pCTLA4hCD59 or BABEpuro vector alone were assayed for cell surface expression of CD59 and CTLA4 by FACS analysis using antibodies to human CTLA4 and human CD59. Briefly, cells were incubated with 10 mg/ml anti CTLA4 (commercially available from Ancell, Bayport, NIN) for 30 min., at 4° C., in 0.1 ml of Dulbecco's phosphate buffered saline (DPBS) containing 1% FBS or bovine serum albumin (BSA). Cells were washed with DPBS before incubation with FITC conjugated antibodies to mouse IgG (commercially available from Zymed, Co., San Francisco, Calif.). FACS analysis was carried out on a Becton Dickenson FACSORT (Becton Dickenson, Franklin Lakes, N.J.) instrument using standard methodologies (Current Protocols in Immunology, ED. J. D. Coligan). The pBABEpuro transduced cells were negative for hCTLA4 expression (FIG. 3). The hCTLA4hCD59 PAEC's exhibited high level expression of hCTLA4 and hCD59 as determined by FACS analysis with antibodies specific to each moiety (FIG. 3 and FIG. 4).

The hCTLA4hCD59 cell line was also treated with PI-PLC and then assayed for expression of the chimeric molecule to further demonstrate that the chimeric molecules were anchored to the cell surface with a CD59 GPI anchor linkage, by enzymatically cleaving the CD59 GPI membrane attachment. FIG. 4 illustrates the loss of cell surface expression following PIPLC treatment, as indicated by reduced antibody reactivity following enzymatic digestion.

### EXAMPLE V

Cell Surface Expression of pCTLA4-hCD59 Chimeric Molecules

Production of a PAEC line to express pCTLA4hCD59 was carried out in the same manner as described in Example IV, however the DNA used to transfect the virus producing cell line was the expression vector pCTLA4hCD59BABEpuro prepared in Example III. FACS analysis for cell surface expression was carried out as described in Example IV. However, detection of the pCTLA4 had to be accomplished by a different method because the human specific anti CTLA4 antibody, ANC152.2, only bound to the human CTLA4 molecule and did not cross react to the pig molecule (data not shown). Therefore, the pCTLA4-hCD59 molecule was detected with the anti CD59 mAb, BRA10G or MEM 43 (Biodesign, Kinnebunk, Me.). FIG. 4 illustrates CD59 expression on hCTLA4-hCD59 and pCTLA4-hCD59 transduced PAEC.

The pCTLA4hCD59 cell line was also treated with PI-PLC and then assayed for expression of the chimeric molecule to demonstrate that the chimeric molecules were anchored to the cell surface with a CD59 GPI anchor linkage by cleaving the CD59 GPI membrane attachment. FIG. 4 illustrates that loss of cell surface expression following PI-PLC treatment. Both moieties could not be detected post digestion.

### EXAMPLE VI

Demonstration of Human CD59 Activity in the hCTLA4-hCD59 Chimera

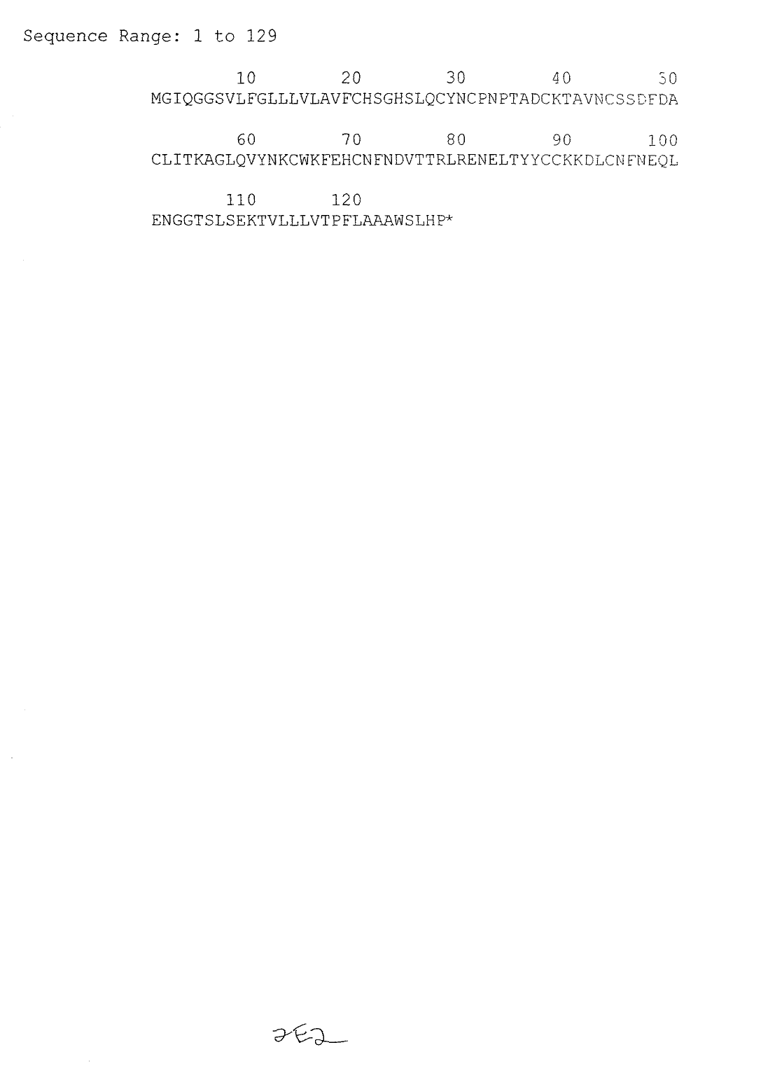

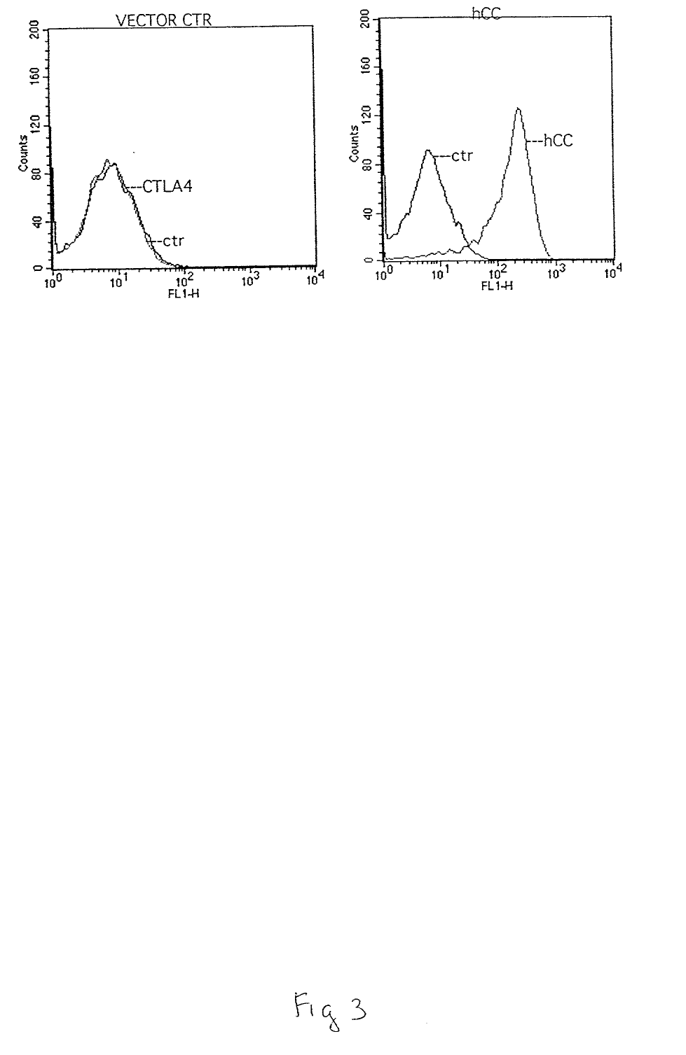

To determine if the CD59 moiety was functional, complement-mediated killing assays were performed using normal human serum as a source of complement. PAECs (5×103) transduced with vector control or hCTLA4hCD59 were seeded into the wells of a flat bottom 96 well plates. After 24 hours adherent cells were washed twice using Hanks balanced salt solution (HBSS) containing 1% BSA (HBSS/BSA). Cells were sensitized by incubating with a polyclonal anti-PAEC antibody (Cocalico, Reamstown, Pa.), followed by incubation with the intracellular dye, Calcein AM (commercially available from Molecular Probes, Eugene, Oreg.) in HBSS/BSA for 30 minutes at 37° C. Excess Calcein AM was removed with two additional washes. Normal human serum (commercially available from Sigma, St. Louis, Mo.) was used as a complement source and was added to a final concentration of 10%, 20% or 40% in 0.05 ml volume diluted in HBSS and the cells were incubated for 1 hour at 37° C. Supernatants containing released calcein from complement lysed cells was transferred to fresh flat bottom microtiter plates. The remaining intact cells with retained calcein were lysed using 0.05 ml 1% sodium dodecylphosphate (SDS). The optical density (OD) at 485 nm was determined for all samples using a cytofluor 2350 spectrophotometer (commercially available from Millipor, Bedford, Mass.). The percent cell death is determined by comparing the OD obtained from untreated cells to that obtained from treated cells. FIG. 5 illustrates the percentage of cell death due to increasing concentrations of human serum. Vector transduced PAECs were susceptible to human serum in a dose dependent manner. However, hCTLA4hCD59 PAECs were 2-3 fold more resistant to human serum induced cell lysis at all concentrations of serum tested as compared to control PAECs

### EXAMPLE VII

Demonstration of Human CTLA4 Activity in the hCTLA4-hCD59 Chimera

When antigen presenting cells (APC) such as PAECs are co-cultured with Jurkat cells, a human T-cell line ATCC TIB152 or human T cells (responder cells) a costimulatory signal results that elicits interleukin 2 (IL-2) production from the responder cells. Therefore, to test the function of the hCTLA4 molecule in the context of the chimeric molecule, costimulation assays were performed using Jurkat cells as responder cells and vector control, hCTLA4hCD59 PAECs or pCTLA4-CD59 as APCs, respectively. The costimulatory capacity of the various PAECs was assayed using a modified endothelial cell costimulation assay (as described in Maher, et al., Journal of Immunology, 157:3838 1999). PAECs transduced with pBABE vector control, hCTLA4hCD59, or pCTLA4-hCD59 were seeded at 5×104 cells per well in 96 well plates (commercially available from Becton Dickenson, Franklin Lakes, N.J.) 24 hours prior to coculturing with T cells. The following reagents were added to final concentrations of 5 ug/ml for antiCD28, or antiB72 (see for example); 10 ug/ml antiCTLA4 (Ancell, Bayport, Minn.); or 5 ug/well sCTLA4Ig (Ancell, Bayport, Minn.). Prior to the costimulation assay, monolayers were washed gently with DPBS three times, followed by the addition of 1×105 Jurkats or T cells as responder cells in 0.09 ml of (spell out RPMI) (RPMI 1640) with FBS, and incubated for 30 minutes at 37° C. PHA, phytohemagglutinin (Sigma L7019) was added in a 0.1 ml volume to a final concentration of 10 mg/ml for 20 hours, at 37° C. Cell free supernatants were collected 20 hours post treatment and assayed for IL-2 by enzyme linked immunosorbent assay (ELISA) (commercially available from R&D Systems, Minneapolis, Minn.). Optical density (OD) at 485 nm was determined using a Microplate Reader 3550 (commercially available from Biorad, Hercules, Calif.) and the OD is proportional to IL-2 production and determined by comparison to a calibration curve generated with known amounts of IL-2. Jurkat supernatants were tested undiluted.

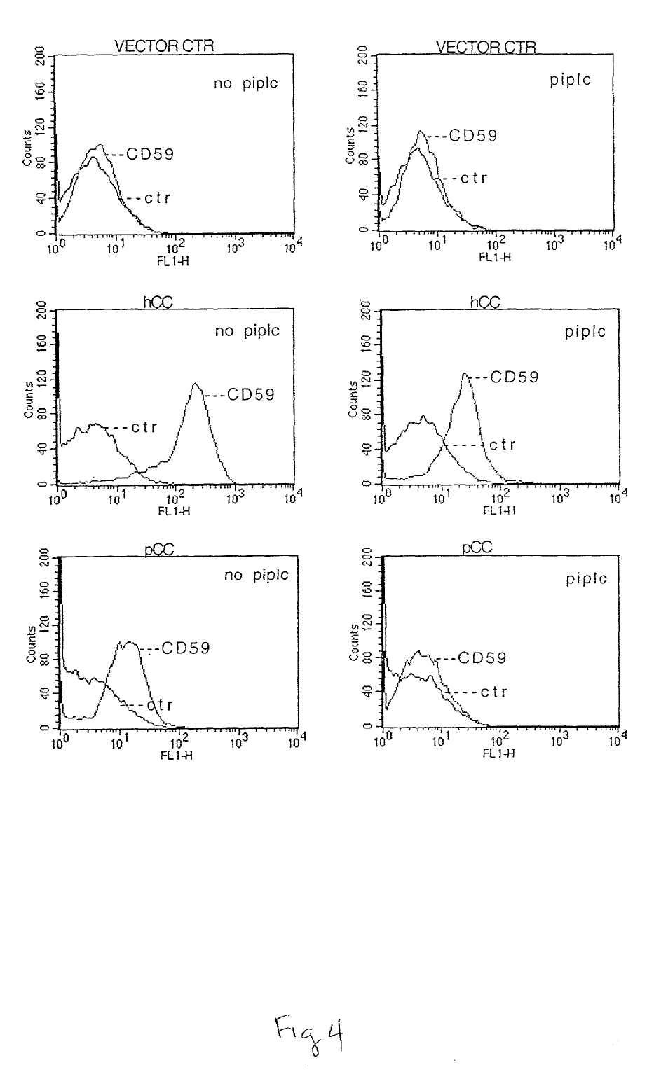

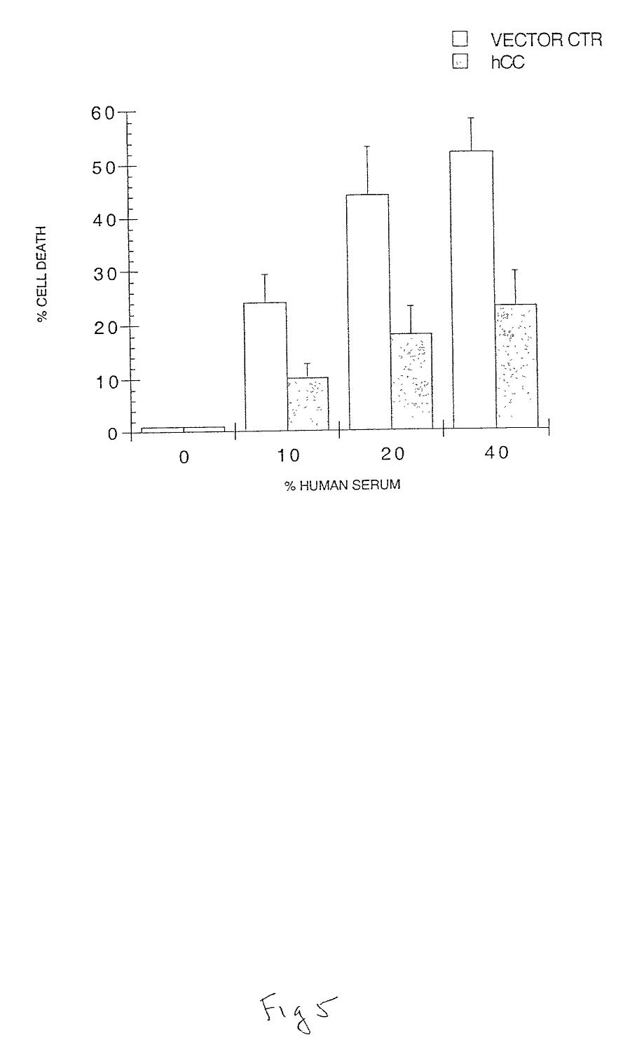

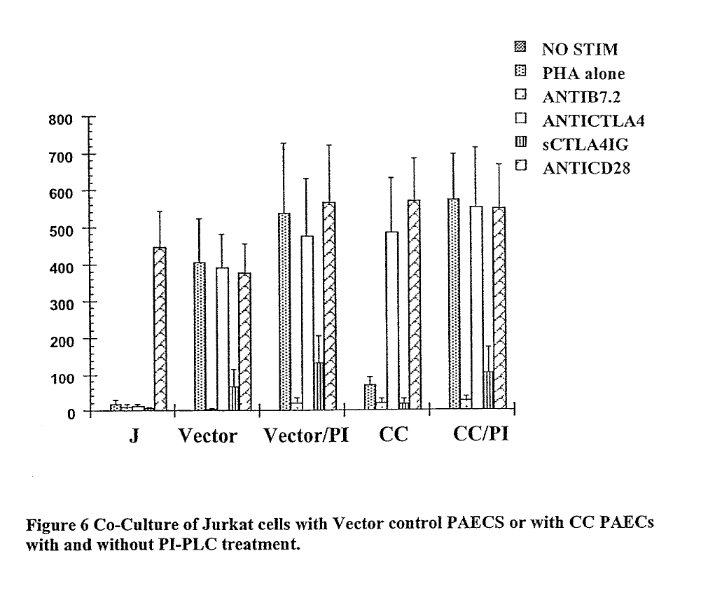

IL-2 release from stimulated Jurkat cells is depicted in FIG. 6. The amount of IL-2 elicited from Junket cells in the presence or absence of pig aortic endothelial cells as antigen presenting cells requires primary and secondary stimulatory signals. Without the secondary co-stimulatory signal provided by an APC or anti CD28, Jurkats remain unactivated, and secrete little to no IL2. The assay utilizes the lectin, phytohemagglutinin (PHA) to cross-link the T cell receptor complex and stimulate the primary signal. When both the primary and secondary signals are provided, 446 pg/ml of IL-2 is secreted. If the secondary signal is provided by vector control PAEC as APC instead of anti CD28, 406 pg/ml IL-2 is secreted. An antibody to pig B7.2 blocks the secondary signal and therefore IL-2 production by specifically binding to the B7.2 molecules on the APC thereby blocking the co-stimulatory second signal. CTLA4 is an alternate ligand for B7.1 and B7.2 and has a ten to twenty fold higher binding affinity than CD28. Therefore, using a soluble form of CTLA4, shCTLA4Ig binds to B7 on PAEC preventing binding of CD28 on Jurkats resulting in no secondary signal. If the secondary signal is provided by an APC bearing the hCTLA4-hCD59, a huge reduction is seen in the secretion of IL-2 to 69 pg/ml, nearly to levels attained with anti pB7.2 or shCTLA4. CTLA4 in the chimeric molecule binds B7.2 in cis preventing Jurkats from CD28 engagement. A blocking antibody to hCTLA4 specifically binds the CTLA4 moiety of the hCTLA4-hCD59 molecule and prevents it from binding to pB7.2 on the CCPAEC surface. B7.2 is therefore available to bind CD28 on Jurkat cells leading to activation and IL-2 secretion. HCTLA4-hCD59 is a gpi linked molecule and, can be cleaved off the cell surface of the cells by phosphotidyl inositol phospholipase C. When hCTLA4-hCD59 is removed the secondary signal is restored, and the Junket cells become activated and secret IL-2.

### REFERENCES

The following references are incorporated herein by reference to more fully describe the state of the art. All patent and literature references cited in the present specification are hereby incorporated by reference in their entirety.

A. Aruffo, B. Seed. 1987. Molecular cloning of a CD28 cDNA by a high-efficiency COS cell expression system. PNAS 84:8573. (cloning CD28)

A. S. B. Edge, M. E. Gosse, J. Dinsmore. 1998. Xenogeneic cell therapy: cur-rent progress and future developments in porcine cell transplantation. Cell Transpl. 7:525.

Adams, et al., 1991. “Contribution of the Repeating Domains of Membrane Cofactor Protein (CD46) of the Complement System to Ligand Binding and Cofactor Activity” Journal of Immunology 147:3005-3011.

Albrecht, et al., 1992. “Herpesvirus Saimiri Has a Gene Specifying a Homologue of the Cellular Membrane Glycoprotein CD59” Virology 190:527-530.

Allen, “Antigen Processing at the Molecular Level”, Immunol. Today 8:270-273 (1987).

Aruffo and Seed, “Molecular Cloning of a CD28 cDNA by a High-Efficiency COS Cell Expression System”, Proc. Natl. Acad. Sci. 84:8573-8577 (1987).

Auchincloss Jr., H., R. Lee, S. Shea, J. S. Markowitz, M. J. Grusby, L. H. Glimcher. 1993. The role of “indirect” recognition in initiating rejection of skin grafts from major histocompatibility complex class 1-deficient mice. PNAS 90:3373.

Auchincloss, H. (1988) Xenogeneic transplantation. A review. Transplantation 46,1-20. Auchincloss, H. (1998) Xenogeneic transplantation. Annu. Rev. Immunol. 16,433-470 B. R. Blazar, P. A. Taylor, A. Panoskaltsis-Mortari, G. S. Gray, D. A. Vallera. 1995. Coblockade of the LFAI:ICAM and CD28/CTLA4:B7 pathways is a highly effective means of preventing acute lethal graft-versus-host disease induced by fully major histocompatibility complex- disparate donor grafts. Blood. 9:2607.

Ausubel, F. M., Brant, R., Kingston, R. E., Moore, D. D., Seidman, J. G., Smith, J. A., Struhl, K., eds., Current Protocols in Molecular Biology. 1991.

Bach, F. H., Turinan, M. A. Vercelloti, G. M., Platt, J. L., and Dalmasso, A. P. (1991) Accommodation: a working paradigm for progressing toward discordant xenografting. Transplant. Proc. 23,205-208.

Brunet et al., “A Differential Molecular Biology Search for Genes Preferentially Expressed in Functional T Lymphocytes: The CTLA Genes”, Immunol. Rev 103:21-36 (1988).

Brunet et al., “A New Member of the Immunoglobulin Superfamily-CTLA-4”, Nature 328:267-270 (1987).

Byme, G. W., McCurry, K. R. Martin, M. J., McClellan, S. M., Platt, J. L., and Logan, J. S. (1997) Transgenic pigs expressing human CD59 and decay accelerating factor produce an intrinsic barrier to complement-mediated daTnage. Transplantation 63, 149-155.

C. A. Bravery, P. Batten, M. H. Yacoub, M. L. Rose. 1995. Direct recognition of SLA- and HLA-like class 11 antigens on porcine endothelium by human T cells results in T cell activation and release of interleukin-2. Transpl. 60:1024.

C. B. Thompson, T. Lindsten, J. A. Ledbetter, S. L. Kunkel, H. A. Young, S. G. Emerson, J. M. Leiden, C. H. June. 1989. CD28 activation pathway regulates the production of multiple T-cell-derived lymphokines/cytokines. PNAS 84:1333. (IL-2 secretion due to CD28)

C. F. Morris, C. J. Simeonovic, M. Fung, J. D. Wilson, A. J. Hapel. 1995. Intragraft expression of cytokine transcripts during pig proislet xenograft rejection and tolerance in mice. J. Immunol. 154:2470. (CD4+T mediate xenorejection)

C. H. June, J. A. Ledbetter, P. S. Linsley, C. B. Thompson. 1990. Role of CD28 receptor in T cell activation. Immunol. Today 11:211.

Chahine, A. A., M. Yu, M. M. McKeman, C. Stoechert, H. T. Lau. 1995. Immunomodulation of pancreatic islet allografts in mice with CTLA41g secreting muscle cells. Transpl. 59:1313.

Chen. C. G., Fisicaro, N., Shinkel, T. A., Aitken, V., Katerelos, M., van Denderen, B. J. W., Tange, M. J., Crawford, R. J., Robins, A. J., Pearse, M. J., and D'Apice, A. J. F. (1996) Reduction in Gal-alpha 1,3-Gal epitope expression in transgenic mice expressing human Htransferase. Xenotransplantation 3, 69-75.

Chen. C. G., Salvaris, E. J., Romanella, M., Katereols, M., Fisicaro, N., Aminian, A., D'Apice, A. J. F., and A. J., Pearse, M. J. (1996) Transgenic expression of human ocl,2fucosyltransferase (H-transferase) prolongs mouse heart survival in an ex vivo model of xenograft rejection. Transplantation 65, 832-837.

Clark et al., “Polypeptides on Human B Lymphocytes Associated with Cell Activation”, Human Immunol. 16:100-113 (1986).

Cohney, S., McKenzie, 1. F. C., Patton, K., Prenzoska, J., Ostenreid, K., Fodor, W. I. Costa, C., Zhao, L., DeCesare, S., and Fodor, W. L. Comparative analysis of three genetic modifications designed to inhibit human serum-mediated cytolysis. Xenotransplantation (in press).

Coligan, J. E., Kruisbeek, A. M. Margulies, D. H., Shevach, E. M., Strober, W. eds. 1992. Current Protocols in Immunology. J. Wiley and Sons, NY.

Cowan, P. J., Chen, C. G., Shinkel, T. A., Fisicaro, N., Salvaris, E., Aminian, A., Romanella, M., Pearse, M. J., and D'Apice, A. J. F. (1998) Knock-out of al,3-galactosyltransferase or expression of cc1,2-fucosyltransfease further protects CD55- and CD59-expressing mouse hearts in an ex vivo model of xenograft rejection. Transplantation 65,1599-1604.

Coyne et al., 1992. “Mapping of epitopes, glycosylation sites, and complement regulatory domains in human decay accelerating factor”, Journal of Immunology 149:2906-2913.

Cozzi, E. Yannoutsos, N., Langford, G. A., Pino-Chavez, G., Wallwork, and White, D. J. G. (1997) Effect of transgenic expression of human decay-accelerating factor on the inhibition of hyperacute rejection of pig organs. In Xenotransplantation (Cooper, D. K. C. and Kemp, E., eds) pp 665-682, Springer-Verlag, Berlin, Heidelberg.

Cramer, D. V. (1996) Natural antibodies. In Transplantation biology: cellular and molecular aspects. (Tilney, N. L., Strom, T. B., Paul, L. C., eds) pp 473-481, Raven Press, New York.

CTLA-4 is a second receptor for the B cell activation antigen B7. J. Exp. Med. 174:561.

Dalmasso, A. P., Vercellotti, G. M., Platt, J. L., and Bach, F. H. (1991) Inhibition of complement-mediated endothelial cell cytotoxicity by decay-accelerating factor. Transplantation 52,530-533.

Dalmasso, A. P., Vercelotti, G. M., Fischel, R. J., Bolman, R. M. Bach, F. H., and Platt, J. L. (1992) Mechanism of complement activation in the hyperacute rejection of porcine organs in primate recipients. Am. J. Pathol. 140, 1157-1166.

Dalmasso, A. P., et al. (1991) “Inhibition of complement-mediated endothelial cell cytotoxicity by decay-accelerating factor” Transplantation 52(3):530-533.

Damle, et al., “Alloantigen-Specific Cytotoxic and Suppressor T Lymphocytes are Derived from Phenotypically Distinct Precursors”, J. Immunol. 131:2296-2300 (1983).

Damle, et al., “Immunoregulatory T Lymphocytes in Man”, J. Immunol. 139:1501-1508 (1987).

Damle, et al., “Monoclonal Antibody Analysis of Human T Lymphocyte Subpopulations Exhibiting Autologous Mixed Lymphocyte Reaction”, Proc. Natl. Acad. Sci. 78:5096-5098 (1981).

Dariavach et al., “Human Ig Superfamily CTLA-4 Gene: Chromosomal Localization and Identity of Protein Sequence Between Murine and Human CTLA-4 Cytoplasmic Domains,” Eur. J. Immunol. 18:1901-1905 (1988).

Daveis, et al., 1989. “CD59, and LY-6-like protein expressed in human lymphoid cells, regulates the action of the complement membrane attack complex on homologous cells” J. Exp. Med. 170:637-654.

Davis, E. A., Pruitt, S. K., Greene, P. S., Ibrahim, S., Lam, T. T., Levin, J. L. Baldwin 111, W. M. Sanfilippo, F. (1996) Inhibition of complement, evoked antibody, and cellular response prevents rejection of pig-to-primate cardiac xenografts. Transplantation 62, 1018-1023.

Diamond, L. E., McCurry, K. R., Martin, M. J., McClellan, Oldham, E. R., Platt, J. L., and Logan, J. S. (1996) Characterization of transgenic pigs expressing functionally active human CD59 on cardiac endothelium. Transplantation 61, 1241-1249.

Fodor, W. L., Williams, B. L., Matis, L. A. Madri, J. A., Rollins, S. A., Knight, J. W., Velander, W., and Squinto, S. P. (1994) Expression of a functional human complement inhibitor in a transgenic pig as a model for the prevention of xenogeneic hyperacute organ rejection. Proc. Natl. Acad. Sci. USA 91, 11153-11157.

Galili, U. (1993) Evolution and pathophysiology of the human anti-a-galactosyl IgG (antiGal) antibody. Springer Semin. ImmunopathoL 15,155-171.

Gallili, U. (1997) Anti-agalasctosyl (Anti-Gal) antibody damage beyond hyperacute rejection. In Xenotransplantation (Cooper, D. K. C. and Kemp, E., eds) pp 95-103, Springer-Verlag, Berlin, Heidelberg.

Goodman, D. J., Millan, M. T., Ferran, C., and Bach, F. H. (1997) Mechanisms of delayed xenograft rejection. In Xenotransplantation (Cooper, D. K. C. and Kemp, E., eds) pp 95-103, Springer-Verlag, Berlin, Heidelberg.

H. Auchincloss, Jr., D. H. Sachs. 1998. Xenogeneic transplantation. Ann. Rev. Immunol. 16:433.

Hancock, W. W., M. H. Sayegh, X. Zheng, R. Peach, P. S. Linsley, L. A. Turka. 1996. Costimulatory function and expression of CD40 ligand, CD80 and CD86 in vascularized murine cardiac allograft rejection. PNAS 93:13967.

Harper, K., C. Balzano, E. Rouvier, M. Mattei, M. Luciani, P. Golstein. 1991. CTLA4 and CD28 activated lymphocyte molecules are closely related in both mouse and human as to sequence, message expression, gene structure, and chromosomal location. J. hmmmol. 147:1037.

Hayes, C. E., and Goldstein, 1. J. (1974) An a-D-galactosyl-binding lectin from Bandeiraea simplicifolia seeds. J. BioL Chem. 249, 1904-1914.

Higgins, P. J., Ko, J. L., Lobell, R., Sardonini, C., Alessi, M. K., Yeh, C. G. 1997. J. 1. 158:287281. A soluble chimeric complement inhibitory protein that possesses both decay-accelerating and factor I cofactor activities.

Inverardi, L., Clissi, B., Stolzer, A. L., Bender, J. R., Sandrin, M. S., and Pardi, R. (1997). Human natural killer lymphocytes directly recognize evolutionary conserved oligosaccharide ligands expressed by xenogeneic tissues. Transplantation 63, 1318-1330.

J. A. Bluestone. 1997. Is CTLA-4 a master switch for peripheral T cell tolerance? J. Immunol. 158:1989.

June et al., “T-Cell Proliferation Involving the CD28 Pathway is Associated with Cyclosporine-Resistant Interleukin 2 Gene Expression”, Mol. Cell. Biol. 7:4472-4481 (1987).

K. Iwata, T. Seya, H. Arita, S. Nagasawa. 1994. Expression of a hybrid complement regulatory protein, membrane cofactor protein decay accelerating factor on Chinese hamster ovary. Comparison of its regulatory effect with those of decay accelerating factor and membrane cofactor protein. J. Immunol. 152:3436.

Kalli, K. R., et al., (1991) “Mapping of the C3b-binding Site of CR1 and Construction of a (CR1).sub.2-F(ab′).sub.2 Chimeric Complement Inhibitor.” J. Exp. Med. 174:14511460. Kennedy, S. P., Rollins, S. A., Burton, W. V., Sims, P. J., Bothwell, A. L. M., Squinto, S. P., and Zavoico, G. B. (1994) Protection of porcine aortic endothelial cells from complement mediated cell lysis and activation by recombinant human CD59. Transplantation 57, 1494-1501.

Kohno et al., “CD28 Molecule as a Receptor-Like Function for Accessory Signals in Cell-Mediated Augmentation of IL-2 Production”, Cell Immunol. 131: 1-10 (1990).

Koike, C., Kannagi, R., Takuma, Y., Akutsu, F., Hayashi, S., Hiraiwa, N., Kadomatsu, T., Muraninatsu, T., Yamakawa, H., Nagai, T., Kobayashi, S., Okada, H., Nakashima, I., Uchida, K., Yokoyama, I., and Takagi, H. (1996) Introduction of cc (1,2)-fticosyltransferase and its effect on (x-Gal epitopes in transgenic pig. Xenotransplantation 3, 81-86.

Kroshus, T. J., Bohnan 111, R. M., Dalmasso, A. P., Rollins, S. A. Guilmette, E. R., Williams, B. L., Squinto, S. P., and Fodor, W. L. (1996) Expression of human CD59 in transgenic pig organs enhances organ survival in an ex vivo xenogeneic perfusion model. Transplantation 61, 1513-1521.

Lafage-Pochitaloff et al., “Human CD28 and CTLA-4 Ig Superfamily Genes are Located on Chromosome 2 at Bands q33-q34,” Immunogenetics 31:198-201 (1990).

Lambrigts, D., Sachs, D. H., and Cooper, D. K. C. (1998) Discordant organ xenotransplantation in primates. Transplantation 66, 547-561.

Larsen, C. P., E. T. Elwood, D. Z. Alexander, S.C. Ritchie, R. Hendrix, C. TuckerBurden, H. R. Cho, A. Aruffo, D. Hollenbaugh, P. S. Linsley, K. J. Winn, T. C. Pearson. 1996. Long-term acceptance of skin and cardiac allografts after blocking CD40 and CD28 pathways. Nature 381:434.

Larsen, R. D., Riverra-Marrero, C. A., Emst, L. K., Cummings, R. D., and Lowe, J. B. (1990) Frameshift and non sense mutations in a human genomic sequence homologous to a murine UDP-Gal:B-D-Gal 1,4-D-GlcNAccd,3-galactosyl transferase cDNA. J BioL Chem. 265, 7055-7061.

Ledbetter et al., “CD28 Ligation in T-Cell Activation: Evidence for Two Signal Transduction Pathways”, Blood 75:1531-1539 (1990).

Ledbetter et al., “Crosslinking of Surface Antigens Causes Mobilization of Intracellular Ionized Calcium in T Lymphocytes”, Proc. Natl. Acad. Sci. 84:1384-1388 (1987).

Lehto, T. et al. (1993) “Interactions of soluble CD59 with the terminal complement complexes” J. Immunol. 151(9):4941-4949.

Lenschow, D. J., Y. Zeng, J. R. Thistlewaite, A. Montag, W. Brady, M. G. Gibson, P. S. Linsley, J. A. Bluestone 1992. Long-term survival of xenogeneic pancreatic islet grafts included by CTLA41g. Sci 257:789.

Lesslauer, et al., “T90/44 (9.3 Antigen), A cell Surface Molecule with a Function in Human T Cell Activation”, Eur. J. Immunol. 16:1289-1296 (1986).

Leventhal, J. R., Dahnasso, A. P., Cromwell, J. W., Platt, J. L., Manivel, C. J., Bolman 111, R. M., and Matas A. J., (1993) Prolongation of cardiac xenograft survival by depletion of complement. Transplantation 55, 857-866.

Leventhal, J. R., John, R., Fryer, J. P., Witson, J. C., Derlich, J., -M., Remiszewski, J., Dalmasso, A. P., Matas, A. J., Bolman 111, R. M. (1995) Removal of baboon and human antiporcine IgG and IgM natural antibodies by immunoabsorption. Transplantation 59,294-300.

Leventhal, J. R., Sakiyalak, P., Witson, J., Simone, P., Matas, A. J., Bohnan, R. M., and Dalmasso, A. P. (1994) The synergistic effect of combined antibody and complement depletion on discordant cardiac xenograft survival in nonhuman primates. Transplantation 57,974-978.

Lin. S. S., Weidner, B. C., Byme, G. W., Diamond, L. E., Lawson, J. H., Hoopes, C. W., Daniels, L. J., Dagget, C. W., Parker, W., Harland, R. C., Davis, R. D., Bollinger, R. R., Logan, J. S., and Platt, J. L. (1998). The role of antibodies in acute vascular rejection of pigto-baboon cardiac transplants. J. Clin. Invest. 101, 1745-1756

Lindsten, T., K. P. Lee, E. S. Harris, B. Petryniak, N. Craighead, P. J. Reynolds, D. B. Lombard, G. J. Freeman, L. M. Nadler, G. S. Gray, C. B. Thompson, C. H. June. 1993. J. hnmunol. 151:3489.

Linsley et al., “Binding of the B Cell Activation Antigen B7 to CD28 Costimulates T Cell Proliferation and Interleukin 2 mRNA Accumulation”, J. Exp. Med. 173:721-730 (1991).

Linsely et al., “T-Cell Antigen CD28 mediates Adhesion with B Cells by Interacting with Activation Antigen B7/BB-1”, Proc. Natl. Acad. Sci. USA, 87:5031-5035 (1990).

Lu, C. Y., Khair-El-Din, T., Dawidson, 1. A., Butler, T. M., Brasky, K. M., Vazquez, M. A., Sicher, S. C. (1994). Xenotransplantation. FASEB J 8, 1122-1130.

Lublin, et al., “Decay-accelerating factor: Biochemistry, molecular biology, and function,” Ann. Rev. Immunol., 7:35-38, 1989.

Lublin, et al., “Molecular cloning and chromosomal localization of human membrane cofactor protein (MCP).” J. Exp. Med., 168:181-194, 1988.

Lublin, et al., “Phospholipid-anchored and Transmembrane Versions of Either Decay-accelerating Factor or Membrane Cofactor Protein Show Equal Efficiency in Protection from Complement-mediated cell Damage,” J. Exp. Med., 174:35-44, 1991.

Marengere, Waterhouse, Duncan, Mittrucker, Feng, T. Mak. 1996. Science 272:1170.

Matsumoto, I., and Osawa, T. (1969) Purification and characterization of an antiH(O) phytohemagglutinin of Ulex europeus. Biochim. Biophys. Acta 194,180-189.

McCurry, K. R., Kooyman, D. L., Alvardo, C. G., Cotterel, A. H., Martin, M. J., Logan, J. S., and Plaft, J., L. (1995) Human complement regulatory proteins protect swine-toprimate cardiac xenografts from humoral injury. Nat. Med. 1, 423-427.

Meri, et al., 1990. “Human protectin (CD590, an 18,000-20,000 MW complement lysis restricting factor, inhibits C5b-8 catalyzed insertion of C9 into lipid bilayers” Immunology 71:1-9.

Miller, A. D., Buttimore, C. Redesign of retrovirus packaging cell lines to avoid recombination leading to helper virus production. Molecular and Cellular biology 6:2895-2902, 1986.

Miyagawa, S., Shirakura, R., lwata, K., Nakata, S., Matsumiya, G., Isutani, H., Matsuda, H., Terado, A., Matsumoto, M., Nagasawa, S., and Seya, T. (1994) Effects of transfected complement regulatory proteins, MCP, DAF, and MCP/DAF hybrid, on complement-mediated swine endothelial cell lysis. Transplantation 58,834-840.

Moran, et al., 1992. “Human recombinant soluble decay accelerating factor inhibits complement activation in vitro and in vivo,” J. Immunol. 149:1736-1743.

Morganstem, J. P., Land, H. Advanced mammalian gene transfer: high titre retroviral vectors with multiple drug selection markers and a complementary helper free packaging cell line. 1990. Nucleic Acid Research. 18:3587.

Norris, et al., 1993. “Structure-Function Relationships of CD59,” Blood, 82(Supple.):202a.

O'Hair, D. P., McManus, R. P., Komorowski, R. (1994) Inhibition of chronic rejection in primate cardiac xenografts using mycophenolate mofetil. Ann. Thorac. Surg. 58, 1311-1315.

Okada, et al., 1989. “Monoclonal antibodies capable of causing hemolysis of neuraminidase-treated human erythrocytes by homologous complement” J. Immunol. 143:2262-2266.

P. M. Wallace, J. S. Johnson, J. F. MacMaster, K. A. Kennedy, P. Gladstone, P. S. Linsley. 1994. CTLA41g treatment ameliorates the lethality of murine graft-versus-host disease across major histocompatibility complex barriers. Transpl. 58:602. (ms is better on ms than hu CTLA41 g).

P. S. Linsley, W. Brady, M. Umes, L. S. Grosmaire, N. K. Dainle, J. A. Ledbetter. 1991.

Pearson, T. C., D. Z. Alexander, K. J. Winn, P. S. Linsley, R. P. Lowry, C. P. Larsen. 1994. Transplantation tolerance induced by CTLA4-1g. Transpl. 57:1701.

Perkins et al., 1988. “A Study of the Structure of Human Complement Component Factor H by Fourier Transform Infared Spectroscopy and Secondary Structure Averaging Methods,” Biochemistry, 27:4004-4012.

Petranka, et al., 1993. “The Structure and Function of CD59: The Importance of the Disulfide Structure and Identification of a Primary Epitope,” Molec. Immunol. 30(suppl. 1):44.

Philbrick, et al., 1990. “The CD59 antigen is a structural homologue of murine Ly-6 antigens but lacks interfron inducibility” Eur. J. Immunol 20:87-92.

Platt, J. L., and Bach, F. H. (1991) Discordant xenografting: challenges and controversies. Curr. Opin. Immun. 3, 735-739.

Pruitt, S. K., Kirk, A. D., Bollinger, R. R., Marsh, H. C. Jr., Collins, B. H., Levin, J. L., Mault, J. R., Heinle, J. S., Ibrahim, S., Rudolph, A. R., Baldwin 111, W. M. and Sanfilippo, F. (1994) The effect of soluble complement receptor type I on hyperacute rejection of porcine xenografts. Transplantation 57, 363-370.

R. H. Schwartz. 1989. Clonal expansion vs. functional clonal inactivation: a costimulatory signaling pathway determines the outcome of T cell antigen receptor occupancy. Annu. Rev. Immunol. 7:445. (Signal two)**

R. H. Schwartz, 1992. Costimulation of T lymphocytes: The role of CD28, CTLA-4, and B7/BB I in interleukin-2 production and immunotherapy. Cell 71:1065. (rev).

Rollins, et al., 1990. “The complement-inhibitory activity of CD59 resides in its capacity to block incorporation of C9 into membrane C5b-9” J. Immunol. 144:3478-3483.

Rollins, et al., 1991, “Inhibition of homologues complement by CD59 is mediated by a species-selective recognition conferred through binding to C8 within C5b-8 or C9 within C5b-9” J. Immunol. 146:2345-2351.

Ross, S. C. , and Densen, P. (1984) Complement deficiency states and infection: epidemiology, pathogenesis and consequences of Neisserial and other infections in an immune deficiency. Medicine 63, 243-273.

Rother, et al., 1994. “Inhibition of Complement-Mediated Cytolysis by the Terminal Complement Inhibitor of Herpesvirus Saimiri,” J. Virol. 68:730-737.

S. A. Rollins, S. P. Kennedy, A. J. Chodera, E. A. Elliott, G. B. Zavoico, L. A. Matis. 1994. Evidence that activation of human T cells by porcine endothelium involves direct recognition of porcine SLA and costimulation by porcine ligands for LFA-1 and CD2. Transpl. 57:1709.

S. E. Maher, K. KarTnann, W. Min, C. C. W. Hughes, J. S. Pober, A. L. M. Bothwell. 1996. Porcine endothelial CD86 is a major costimulator of xenogeneic human T cells. J. Immunol. 157:3838.

Saadi, S., and Platt, J. L. (1995) Transient perturbations of endothelial integrity induced by natural antibodies and complement. J. Exp. Med. 181, 21-31.

Sambrook, J., Fritsch, E. F., and T. Maniatis. 1989. Molecular Cloning: A laboratory manual. 2^{nd} edition. Cold Spring Harbor Press.

Sandrin, M. S. (1997) Down-regulation of Galcc(1,3)Gal expression by alpha 1,2fucosyltransferase. Further characterization of alpha 1,2-fucosyltransferase transgenic mice. Transplantation 64, 495-500.

Sandrin, M. S., Fodor, W. L., Mouhtouris, E., Osman, N., Cohney, S., Rollins, S. A., Guilmette, E. R., Setter, E., Squinto, S. P., and McKenzie, 1. F. C. (1995) Enzymatic remodeling of the carbohydrate surface of a xenogenic cell substantially reduces human antibody binding and complement-mediated cytolysis. Nature Med. 1, 1261-1267.

Sandrin, M. S., Vaughan, H. A., Dabkowski, P. L., and McKenzie, 1. F. C. (1993) Anti-pig IgM antibodies in human serum reacts predominantly with Gal(XI,3)Gal epitopes. Proc. Natl. Acad. Sci. USA 90,11391-11395.

Sawada, et al., 1990. “Isolation and expression of the full-length CDNA encoding CD59 antigen of human lymphocytes”. DNA and Cell. Biol. 9: 213-220.

Schaapherder, A. F. M., Daha, M. R., Te Bulte, M. -T. J. W., Van der Woude, F. J., and Gooszen, H. G. (1994) Antibody-dependant cell-mediated cytotoxicity against porcine endothelium induced by a majority of human sera. Transplantation 57, 1376-1382.

Schwartz, “A Cell Culture Model for T Lymphocyte Clonal Anergy”, Science 248:1349-1356 (1990).

Sharma, A., Okabe, J., Birch, P., McClellan, S. B., Martin, M, J., Platt, J. L., and Logan, J. S. (1996) Reduction in the level of Galcc(1,3)Gal in transgenic mice and pigs by expression of an (x(1,2)fucosyltransferase. Proc. NatL Acad. Sci. USA 93, 7190-7195.

Sommerville, C. A., and D'Apice, A. J. F. (1993) Future directions in transplantation: xenotransplantation. Kidney International 44, Suppl. 42, S I 12-S 121.

Southern, E. M. (1975) Detection of specific sequences among DNA fragments separated by gel electrophoresis. J MoL BioL 98, 503-517.

Stefanova, et al., 1989. “Characterization of a broadly expressed human leucocyte surface antigen MEM-43 anchored in membrane through phosphatidylinositol” Mol. Immunol. 26:153-161.

Storb and Thomas, “Graft-Versus-Host Disease in Dog and Man: The Seattle Experience”, Immunol. Rev. 88:215-238 (1985).

Su, et al., 1991. “The Glycosyl Phosphatidylinositol Anchor Is Critical for Ly-6A/E-mediated T Cell Activation,” J. Cell Biol. 112:377-384.

Sun, H. V. Subbotin, C. Chen, A. Aitouche, L. A. Valdivia, M. H. Sayegh, P. S. Linsley, J. J. Fung, T. E. Starzl, A. S. Rao. 1997. Prevention of chronic rejection in mouse aortic allografts by combined treatment with CTLA4-Ig and anti-CD40 ligand monoclonal antibody Transpl. 64:1838.

Thompson et al., “CD28 Activation Pathway Regulates the Production of Multiple TCell-Derived Lymphokines/Cytokines”, Proc. Natl. Acad. Sci. 86:1333-1337 (1989).

Tone, et al., 1992. “Gene structure of human CD59 and demonstration that discrete mRNAs are generated by alternative polyadenylation” J. Mol. Biol. 227:971-976.

Tran, H. M., P. W. Nicherson, A. C. Restifo, M. A. Ivis-Woodward, A. Patel, R. D. Allen, T. B. Strom, P. J. O'Connell. 1997. Distinct mechanisms for the induction and maintenance of allograft tolerance with CTLA4-fc treatment. J. Immunol. 159:2232.

Vanhove, B., deMartin, R., Lipp, J., and Bach, F. H. (1994) Human xenoreactive natural antibodies of IgM isotype activate pig endothelial cells. Xenotransplantation 1, 17-22.

Venneker, et al., 1992. “CD59: a molecule involved in antigen presentation as well as downregulation of membrane attack complex” Exp. Clin. Immunogenet. 9:33-47.

W. L. Fodor, S. A. Rollins, E. R. Guilmette, E. Setter, S. P. Squinto. 1995. A novel bifunctional chimeric complement inhibitor that regulates C3 convertase and fon-nation of the membrane attack complex. J. Immunol 155:4135.

W. Steurer, P. W. Nickerson, S. W. Steele, J. Steiger, X. X. Zheng, T. B. Strom 1995. Ex vivo coating of Islet cell allografts with murine CTLA4/fc promotes graft tolerance. J. Immunol. 155:1165.

Walsh, et al., 1991. “Transfection of human CD59 complementary DNA into rat cells confers resistance to human complement” Eur. J. Immunol. 21:847-850.

Walunas, T. L., D. J. Lenschow, C. Y. Bakker, P. S. Linsley, G. J. Freeman, J. M. Green, C. B. Thompson, J. A. Bluestone. 1994. CTLA-4 can function as a negative regulator of T cell activation. Immunity 1:405.

Waterhouse, P., J. M. Penninger, E. Timms. A. Wakeham, A. Shahinian, K. P. Lee, C. B. Thompson, H. Griesser, T. W. Mak. 1995. Lymphoproliferative disorders with early lethality in mice deficient in CTLA-4. Sci 270:985.

Weaver and Unanue, “The Costimulatory Function of Antigen-Presenting Cells”, Immunol. Today 11:49-55 (1990).

Weiss, “Structure and Function of the T Cell Antigen Receptor”, J. Clin. Invest. 86:1015-1022 (1990).

Whitlow, et al., 1990. “H19, a surface membrane molecule involved in T-cell activation, inhibits channel formation by human complement” Cell. Immunol. 126:176-184.

Wing, et al., 1992. “Oligodendrocytes lack glycolipid anchored proteins which protect them against complement lysis. Restoration of resistance to lysis by incorporation of CD59”. Immunology 76:140-145.

Wm. Paul. 1998. Fundamental Immunology. Fourth Edition.

Yockochi et al., “B Lymphoblast Antigen (BB-1) Expressed on Epstein-Barr VirusActivated B Cells Blasts, B Lymphoblastoid Cell Lines, and Burkitt's Lymphomas”, J. Immunol. 128:823:827 (1981).

Zhao, et al., 1991. “Amplified gene expression in CD59-transfected Chinese hamster ovary cells confers protection against the membrane attack complex of human complement” J. Biol. Chem. 266: 13418-13422.

It will be understood that various modifications may be made to the embodiments disclosed herein. For example, the C5b-9 inhibitory domain and/or the T Cell inhibitory domain may be modified by creating amino acid substitutions or nucleic acid mutations provided at least some complement regulatory activity and some T Cell inhibitory activity remains after such modifications. Similarly, the nucleotide sequences of the chimeric protein, protein may be modified by creating nucleic acid mutations which do not significantly change the encoded amino acid sequences, including third nucleotide changes in degenerate codons (and other “silent” mutations that do not change the encoded amino acid sequence). Mutations which result in a highly conservative or silent amino acid substitution for an encoded amino acid while leaving the characteristics of the chimeric proteins essentially unchanged are also within the scope of disclosure. Also included are sequences comprising changes that are found as naturally occurring allelic variants of the genes for the T Cell inhibitory molecules and the C5b-9 inhibitory molecules used to create chimeric molecules described herein. All of the foregoing shall be considered as equivalents of the specific embodiments set forth herein. Therefore, the above description should not be construed as limiting, but merely as exemplifications of preferred embodiments. Those skilled in the art will envision other modifications within the scope of the claims appended hereto.

What is claimed is:

1. A chimeric protein capable of inhibiting both cellular and humoral immune responses.

2. A chimeric protein comprising a domain having C5b-9 inhibitory activity and a domain having T Cell inhibitory activity.

3. A chimeric protein according to claim 2, wherein the protein exhibits at least about 25% of the C5b-9 inhibitory activity of said naturally occurring C5b-9 inhibitor protein.

4. A chimeric protein according to claim 2, wherein the protein has at least about 25% of the T Cell inhibitory activity of a naturally occurring T Cell inhibitor protein.

5. A chimeric protein according to claim 2, wherein the domain having C5b-9 inhibitory activity is derived from mammalian CD59.

6. A chimeric protein according to claim 2, wherein the domain having T Cell inhibitory activity is derived from mammalian CTLA4.

7. A chimeric protein according to claims 6, wherein the mammalian CTLA4 is selected from the group consisting of human and porcine CTLA4.

8. A chimeric protein according to claim 2, wherein the protein includes a linker region between the domain having C5b-9 inhibitory activity and the domain having T Cell inhibitory activity.

9. A chimeric protein according to claim 2, further comprising a cellular anchor moiety.

10. A chimeric protein according to claim 9, wherein the cellular anchor moiety is a GPI anchor.

11. A chimeric DNA construct comprising a domain derived from a DNA sequence encoding a domain having C5b-9 inhibitory activity and a DNA sequence encoding a domain having T Cell inhibitory activity.

12. A chimeric DNA construct according to claim 11, wherein the DNA sequence encoding a domain having C5b-9 inhibitory activity is derived from a DNA sequence encoding CD59.

13. A chimeric DNA construct according to claim 11, wherein the DNA sequence encoding a domain having T Cell inhibitory activity is derived from a DNA sequence encoding mammalian CTLA4.

14. A chimeric DNA construct according to claim 11, wherein the mammalian CTLA4 is selected from the group consisting of human and porcine CTLA4.

15. A cloning vector comprising a DNA construct according to claim 11.

16. A cloning vector according to claim 15, wherein the cloning vector is a retroviral vector.

17. A host cell transformed by the vector of claim 15.

18. Transgenic porcine cells comprising the chimeric protein of claim 10.

19. Transgenic porcine tissues comprising the chimeric protein of claim 10.

20. Transgenic whole organs comprising the chimeric protein of claim 10.

21. A chimeric protein comprising a domain having C3 inhibitory activity and a domain having T Cell inhibitory activity.

22. A chimeric protein according to claim 21, wherein the domain having C5b-9 inhibitory activity is derived from mammalian DAF.
